(19) United States
(12) Patent Application Publication
(10) Pub. No.: US 2014/0286926 A1
(43) Pub. Date: Sep. 25, 2014

(54) METHOD FOR TREATMENT OR PREVENTION OF ALLERGIC DISEASES

(71) Applicant: ProMD Biotech, Co., Ltd., Tainan City (TW)

(72) Inventors: Wei-Chih Su, Tainan City (TW); Hsiang-Ling Chen, Tainan City (TW); Chun-Hsien Huang, Tainan City (TW); Hsiao-Li Wu, Tainan City (TW); Kuang-Chih Lee, Tainan City (TW); Pei-Yu Tsai, Tainan City (TW); Chun-Fu Tseng, Tainan City (TW)

(73) Assignee: ProMD Biotech, Co., Ltd., Tainan City (TW)

(21) Appl. No.: 14/223,771

(22) Filed: Mar. 24, 2014

(30) Foreign Application Priority Data
Mar. 22, 2013 (TW) 102110344
Mar. 21, 2014 (TW) 103110733

(51) Int. Cl. A61K 38/44; A23L 1/305; A61K 35/74

(52) U.S. Cl. CPC A61K 38/44; A61K 35/742; A23L 1/3058; C12Y 102/01012
USPC 424/94.4

(57) ABSTRACT

The present invention provides a method for reducing an allergic response and treating or preventing an allergic disease, comprising administering a subject in need thereof a therapeutically effective amount of the active ingredient for the treatment or the prevention of allergic diseases, wherein the active ingredient is glyceraldehyde-3-phosphate Dehydrogenase (G3PDH) or the functional variant or fragment thereof. The G3PDH can be isolated from Lactobacillus gasseri PM-A0005 (deposited under Budapest Treaty in the China Center for Type Culture Collection (CCTCC) with Deposit No: M 207039), as well as extract, fraction, and sub-fraction thereof.

### FIELD OF THE INVENTION

The present invention relates to a method for preventing or treating an allergic disease. In particular, the present invention relates to a method or composition for preventing or treating and allergic disease, such as asthma or atopic dermatitis by reducing an allergic response.

### BACKGROUND OF THE INVENTION

Allergic diseases such as allergic atopic dermatitis, urticarial, repeated episodes of allergic rhinitis, and allergic asthma have been serious social problems in developed countries. A well-known hypothesis is that the smaller the chance for an infant to be exposed to immune stimulating pathogens, the higher the possibility for him to be infected with an allergy-related disease. Correlation studies have indicated that when allergic diseases occur, the immune response in human body reduces the amount of Th1 cells, and generates several types of cytokines to stimulate the immune response towards the Th2 pathway. Thus, the allergic diseases activate the humoral immune response, and generate immunoglobulin E (IgE) and an excess of eosinophil. Regulating and balancing the Th2 immune response caused by allergies through the Th1 immune response can improve a person's susceptibility to allergies.

Treatment for asthma still mainly consists of medication, such as steroids and drugs, which inhibit the release of inflammatory substances in mast cells (e.g. bronchodilator, immunotherapy, etc.). However, drug therapy including anti-inflammatory drugs and bronchodilator, is only a temporary method, seeing how it cannot truly change allergic immune responses. On the other hand, desensitization can change a person's susceptibility to allergies, but the treatment usually takes place over a long period of time. Additionally, desensitization occasionally has side effects, and is not appropriate for all patients.

Therefore, the development of an active ingredient for an anti-allergy drug based on the mechanism of stimulating immune cells and regulating the Th1/Th2 immune response is still needed.

### SUMMARY OF THE INVENTION

In the present invention, it is unexpectedly found that glyceraldehyde-3-phosphate dehydrogenase (G3PDH) has an effect in preventing and treating allergic diseases.

Accordingly, in one aspect, the present invention provides a method for preventing or treating an allergic disease in a subject. The method comprises administering the subject with a composition comprising a therapeutically effective amount of glyceraldehyde-3-phosphate Dehydrogenase (G3PDH) or the functional variant or fragment thereof as an active ingredient to reduce allergic response.

In one example of the invention, the allergic disease is asthma or atopic dermatitis.

In one example of the invention, the G3PDH has the amino acid sequence set forth in SEQ ID No: 1.

According to the present invention, the G3PDH can be obtained by isolating from Lactobacillus gasseri PM-A0005 strain (PM-A0005 strain), the extract, fraction, or sub-fraction thereof. The PM-A0005 strain was deposited in the China Center for Type Culture Collection (CCTCC) with Deposit No: M 207039.

In another aspect, the present invention provides a method for treating or preventing atopic dermatitis in a subject comprising administering the subject with a composition comprising a therapeutically effective amount of G3PDH or the functional variant or fragment thereof as an active ingredient.

In one further aspect, the present invention provides a method for treating or preventing atopic dermatitis in a subject comprising administering the subject with a composition comprising a therapeutically effective amount of the PM-A0005 strain, the extract, fraction, or sub-fraction thereof.

In one example of the invention, the composition comprises the PM-A0005 strain. In another example of the invention, the composition comprises the extract of the PM-A0005 strain.

It is believed that a person of ordinary skill and knowledge in the art to which the present invention belongs can utilize the present invention to its broadest scope based on the descriptions herein with no need of further illustration. Therefore, the following descriptions should be understood to be for demonstrative purpose only, and do not limit the scope of the present invention in any way.

### BRIEF DESCRIPTION OF THE DRAWINGS

The foregoing summary, as well as the following detailed description of the invention, will be better understood when read in conjunction with the appended drawing. In the drawings:

FIGS. 1A-1C show the results of the further isolation of the active ingredient from PM-A0005 strain extract with ion-exchange chromatography. FIG. 1A shows the condition setting of ion-exchange chromatography. FIG. 1B shows that extract AS_{—}0-25% can isolate 3 fractions. FIG. 1C shows that extract AS_{—}50-75% also can isolate 3 fractions.

FIGS. 2A-2E show the results of further isolation of the active ingredient from the fractions, IE1-2, IE1-3, IE3-2, and IE3-3, with gel-filtration chromatography. FIG. 2A shows the condition setting of gel-filtration chromatography. FIG. 2B shows that the fraction IE1-2 can isolate 1 sub-fraction, IE1-2G1. FIG. 2C shows that IE1-3 can isolate 2 sub-fractions, IE1-3G1 and IE1-3G2. FIG. 2D shows that IE3-2 can isolate 3 sub-fractions, IE3-2G1, IE3-2G2, and IE3-2G3. FIG. 2E shows that IE3-3 can isolate 5 sub-fractions, IE3-3G1, IE3-3G2, IE3-3G3, IE3-3G4, and IE3-3G5.

FIG. 3 shows the result of inducing bronchoconstriction, with methacholine, and measuring mice airway resistance after intraperitoneally injected the extract AS_{—}50-75% (Der p-ip-AS) and the sub-fraction IE3-3G1 (Der p-ip-IE) to mice, wherein the airway resistance is represented as Penh value.

FIGS. 4A-4C show the evaluations of lung tissue change in Der p allergen-sensitized mice, demonstrating that the administration of extract AS_{—}50-75% and sub-fraction IE3-3G1 can significantly reduce inflammation and cell infiltration in lungs. FIG. 4A shows the result of the cell number calculation in lung cell irrigant. FIG. 4B shows the cell classification of lung cell irrigant. FIG. 4C shows the concentration of thymus and activation-regulated chemokine (TARC) in lung cell irrigant, wherein the extract AS_{—}50-75% is administered to Der p-ip AS, and the sub-fraction IE3-3G1 is administered to Der p-ip-IE.

FIG. 5 shows the result of H&E staining the lung tissue in Der p allergen-sensitized mice, where the administration of the extract AS_{—}50-75% and the sub-fraction IE3-3G1 can effectively reduce the thickening of the airway wall and improve the number of immune cells infiltration in mice, respectively. Wherein the control is naïve (FIG. 5A), and the Der p allergen-sensitized group is sensitized with Der p (FIG. 5B), Der p-ip-AS is administered with extract AS_{—}50-75% (FIG. 5C) and Der p-ip-IE is administered with the sub-fraction IE3-3G1 (FIG. 5D).

FIGS. 6A-6D show the results of the oral treatment with 1×10⁷ CFU or 1×10⁹ CFU of PM-A0005 strain rescued physiological features of atopic dermatitis. FIG. 6A shows the skin appearance of SKH1 mice. FIG. 6B shows the hydration of skin which was measured by Cutometer® MPA. FIG. 6C shows the pH value of skin which was measured by Cutometer® MPA. FIG. 6D shows the trans-epidermal water loss (TEWL) of skin which was measured by Tewameter TM210. Data represent the mean±SD (n=4). *P<0.05, **P<0.01, ***P<0.001, paired t-test.

FIGS. 7A-7D shows the skins of atopic dermatitis mice with intraperitoneal (i.p.) injection IE3-3G1 were improved comparing with PBS treatment. FIG. 7A shows the skin appearance of SKH1 mice. FIG. 7B shows the hydration of skin which was measured by Cutometer® MPA. FIG. 7C shows the pH value of skin which was measured by Cutometer® MPA. FIG. 7D shows the trans-epidermal water loss (TEWL) of skin which was measured by Tewameter TM210. Data represent the mean±SD (n=4). *P<0.05, **P<0.01, ***P<0.001, paired t-test.

FIGS. 8A-8C show the results of oral treatment with 1×10⁷ CFU or 1×10⁹ CFU PM-A0005 strain decreased skin thickness and eosinophils infiltration in atopic dermatitis mice. FIG. 8A shows the histological H&E staining in sections from sensitized skin sites. FIG. 8B shows the thickness of skin which was measured in H&E staining sections. FIG. 8C shows the numbers of eosinophils per view. Data represent the mean±SD (n=6). *P<0.05, **P<0.01, ***P<0.001, paired t-test.

FIGS. 9A-9C show the results of the skin thickness and eosinophils infiltration decreased in atopic dermatitis mice with IE3-3G1 treatment. FIG. 9A shows the histological H&E staining in sections from sensitized skin sites. FIG. 9B shows the thickness of skin which was measured in H&E staining sections. FIG. 9C shows the numbers of eosinophils per view. Data represent the mean±SD (n=6). *P<0.05, **P<0.01, ***P<0.001, paired t-test.

FIG. 10 shows the results of the oral treatment with 1×10⁷ CFU or 1×10⁹ CFU L. gasseri (PM-A0005) decreased TSLP expression in atopic dermatitis mice. Sections from sensitized skin sites were stained with TSLP antibody and reacted with DAB.

FIGS. 11A and 11B show the decreased Langerhans's cells in skin of atopic dermatitis mice after oral treatment with 1×10⁷ CFU or 1×10⁹ CFU of the PM-A0005 strain.

FIG. 11A shows the results of sections from sensitized skin sites were stained with Langerin antibody and reacted with AEC. FIG. 11B shows the numbers of Langerhans cells in epidermis or dermis per view. Data represent the mean±SD (n=6). *P<0.05, **P<0.01, ***P<0.001, paired t-test.

FIG. 12 shows the result of the TSLP expression decreased in atopic dermatitis mice with IE3-3G1 treatment. Sections from sensitized skin sites were stained with TSLP antibody and reacted with DAB.

FIGS. 13A and 13B show the decreased numbers of Langerhans's cells in skin of atopic dermatitis mice with IE3-3G1 treatment. FIG. 13A shows the results of sections from sensitized skin sites were stained with Langerin antibody and reacted with AEC. FIG. 13B shows the numbers of Langerhans cells in epidermis or dermis per view. Data represent the mean±SD (n=6). *P<0.05, **P<0.01, ***P<0.001, paired t-test.

FIGS. 14A and 14B show the effects of total IgE and antigen-specific IgE in atopic dermatitis mice after oral treatment with 1×10⁷ CFU or 1×10⁹ CFU of the PM-A0005 strain. FIG. 14A shows the total IgE levels in four groups. FIG. 14B shows the OVA-specific IgE levels in four groups. Data represent the mean±SD (n=6). *P<0.05, **P<0.01, ***P<0.001, paired t-test.

FIGS. 15A-15C show the effects of total IgE and antigen-specific IgE in atopic dermatitis mice with IE3-3G1 treatment. FIG. 15A shows the total IgE levels in four groups. FIG. 15B shows the OVA-specific IgE levels in four groups. FIG. 15C shows the Der p-specific IgE levels in four groups. Data represent the mean±SD (n=6). *P<0.05, **P<0.01, ***P<0.001, paired t-test.

FIG. 16 shows the result of the oral treatment with 1×10⁷ CFU or 1×10⁹ CFU of the PM-A0005 strain had no effect on proliferation ratio of splenocytes in atopic dermatitis mice. Splenocytes cultured with PHA-L or OVA for 72 hr and the proliferation ratio was determined by Cell Counting Kit-8 assay. Data represent the mean±SD (n=6). *P<0.05, **P<0.01, ***P<0.001, paired t-test.

FIGS. 17A and 17B show the results of treatment with IE3-3G1 decreased proliferation ratio of splenocytes in Der p sensitization mice but had no effect on OVA sensitization mice. Splenocytes cultured with PHA, OVA, or Der p for 72 hrs and the proliferation ratio was determined by Cell Counting Kit-8 assay. Data represent the mean±SD (n=6). *P<0.05, **P<0.01, ***P<0.001, paired t-test.

FIGS. 18A-18C show the change of cytokine profiles in atopic dermatitis mice treated with 1×10⁷ CFU or 1×10⁹ CFU of the PM-A0005 strain orally. Splenocytes cultured with PHA-L and OVA for 48 hr, and the results were shown as FIG. 18A: IFN-γ, FIG. 18B: IL-17, and FIG. 18C: IL-10. Data represent the mean±SD (n=6). *P<0.05, **P<0.01, ***P<0.001, paired t-test.

FIGS. 19A-19C show the change of cytokine profiles in atopic dermatitis mice with IE3-3G1 treatment. Splenocytes cultured with PHA-L, OVA, or Der p for 48 hr, and the results were shown as FIG. 19A: IFN-γ, FIG. 19B: IL-17, and FIG. 19C: IL-10. Data represent the mean±SD (n=6). *P<0.05, **P<0.01, ***P<0.001, paired t-test.

FIG. 20 shows the result of the oral treatment with 1×10⁷ CFU or 1×10⁹ CFU of the PM-A0005 strain decreased IL-17 expression in skin. Sections from sensitized skin sites were stained with IL-17 antibody and reacted with DAB.

FIG. 21 shows the result of the treatment with IE3-3G1 decreased IL-17 expression in skin. Sections from sensitized skin sites were stained with IL-17 antibody and reacted with DAB.

FIG. 22 shows the result of the protein two-dimensional electrophoresis analysis of the sub-fraction IE3-3G1. Number 1-5 are G3PDH; Number 6 is fructose diphosphate aldolase.

FIG. 23 shows the identification of G3PDH recombinant protein through a Western Blot analysis, using Coomassie Blue staining and Histidine antibody. Wherein M is the protein molecular marker; 1 is the sub-fraction IE3-3G1; and, 2 is the G3PDH-His recombinant protein.

FIG. 24 shows the evaluation of immune regulation effectiveness with G3PDH recombinant protein stimulating mice dendritic cells to generate cytokine IL-12 (IL 12p40), wherein the additions of LPS and PHA are the positive control, and the control group is the cells only, with no additional stimulus.

FIG. 25 shows the evaluation of immune regulation effectiveness with G3PDH recombinant protein stimulating mice spleen cells to generate cytokine Interferon-γ (IFN-γ), wherein additions of LPS and PHA are the positive control, and the control group is the cells only, with no additional stimulus.

### DETAILED DESCRIPTION OF THE INVENTION

Unless defined otherwise, all technical and scientific terms used herein have the same meaning as commonly understood by a person skilled in the art to which this invention belongs. As used herein, the following terms have the meanings ascribed to them unless specified otherwise.

As used herein, the singular forms “a”, “an”, and “the” include plural referents unless the context clearly dictates otherwise. Thus, for example, reference to “a sample” includes a plurality of such samples and equivalents known to those skilled in the art.

As used herein, the term “allergic disease” refers to any disease triggered by the interaction of an allergen, allergen-specific immunoglobulin E, mast cell, etc. The allergen binds to immunoglobulin E (IgE), attached in the fragment constant (Fc) receptor of the mast cell, and then triggers the mast cell to degranulation. Some mediums released by the granules, such as histamine, leukotriene and chemotactic factor, further increase of the permeability in blood vessels, the contraction of the trachea, cause other symptoms, and simultaneously attract other cells (e.g. neutrophil and eosinophil) which lead to an inflammatory response, causing the chronic inflammation of skin, mucosal tissue, or blood vessel. Allergic diseases include, but are not limited to, allergic rhinitis, atopic dermatitis and asthma, and allergies caused by specific foods and insect bites, resulting in a severe inflammatory response and giving rise to the chronic inflammation of skin, mucosal tissue, or blood vessel.

As used herein, the term “asthma” refers to an uncontrollable airway hyper-responsiveness due to the inhalation of an inducible allergen, accompanied by a change in the structure of the airway, including, but not limited to, epithelial hyperplasia, deformation of the mucosa, airway smooth muscle proliferation, and hyperplasia of the extracellular matrix.

As used herein, the term “atopic dermatitis” refers to a type of dermatitis, an inflammatory, relapsing, non-contagious and itchy skin disorder, including, but not limited to, neurodermitis, endogenous eczema, flexural eczema, and infantile eczema.

As used herein, the term “functional variants” refers to the polypeptide obtained from using methods such as recombinant DNA technology, through insertion, deletion, or substitution by one or more amino acids that is different from the known protein amino acid sequence, but does not affect the function thereof. For example, amino acid substitutions are created due to the replacement of an amino acid based on another similar structure and/or the chemical properties of the amino acid (e.g. conservative amino acid replacement). Conservative amino acid substitutions can be based on residue polarity, charge, solubility, hydrophobicity, hydrophilicity and/or the amphipathic nature of similarity. An inserted amino acid or amino acid deletion preferably falls within 1-20 amino acids, i.e. more preferably 1-10 amino acids. Through recombinant DNA technology, the insertion, deletion, or replacement, and testing allows the use of the resulting recombinant variants in the same function of the original amino acid. As used herein, the term “functional fragment” refers to sections of the amino acid, wherein the fragments provide the same function in known proteins or functional variants. For example, the amino acid sequence of the epitope.

As used herein, the term “acceptable” refers to the fact that the carrier must be compatible with the active ingredient of the composition (preferably stabilizes the active ingredient) and is harmless to the subject treated.

As used herein, the term “therapeutically effective amount” refers to the amount necessary for the dose of the drug or pharmaceutical to cause the desired pharmacological or physiological result, or the amount necessary for the treatment, cure, prevention, or improvement of the disease, disorder, or side effects, or the amount necessary to reduce the occurrence of disease or disorder. The effective amount or effective dose depends on the specific active ingredient, the mode of administration, age, body weight, and the condition of the treated subject. The precise amount of the pharmaceutical is administered based on the judgment of the doctor and differences in the treated subjects.

According to the present invention, it is unexpectedly found that glyceraldehyde-3-phosphate dehydrogenase (G3PDH) has anti-allergic activity. In one embodiment of the present invention, G3PDH was co-cultured with mice dendritic cells, and the experimental results demonstrated that G3PDH is effective in inducing mice dendritic cells to express IL-12 and stimulating spleen cells to express IFN-γ. Thus, G3PDH is effective in stimulating immune cells and regulating immune responses. Therefore, the present invention provides a method for preventing or treating an allergic disease in a subject comprising administering the subject with a composition comprising a therapeutically effective amount of glyceraldehyde-3-phosphate Dehydrogenase (G3PDH) or the functional variant or fragment thereof as an active ingredient to reduce allergic response.

In one embodiment of the present invention, G3PDH has the amino acid sequence set forth in SEQ ID No: 1. However, the G3PDH used in the present invention shall include G3PDH from different sources and processes and the functional variant or fragment thereof.

According to the present invention, G3PDH may be isolated from Lactobacillus gasseri PM-A0005 (PM-A0005 strain), which was deposited under Budapest Treaty in the China Center for Type Culture Collection (CCTCC) with Deposit No: M 207039. Furthermore, G3PDH may be isolated from the extract, fraction, sub-fraction of the PM-A0005 strain.

According to the present invention, the extract of the PM-A0005 strain can be obtained by the standard method known in the art, such as decomposition of the strain by lysozyme and the precipitation of the cytoplasmic protein by ammonium sulfate. In one example of the present invention, the extract may be obtained by decomposing the culture of the PM-A0005 strain by lysozyme and precipitating the cytoplasmic protein by ammonium sulfate. In one example, the ammonium sulfate is in a concentration more than 0% but less than 25%, or a concentration of 50-75%.

In one example of the present invention, the fraction may be obtained by further purification through ion-exchange chromatography, such as Sephacryl™ S-300 HR column. In one example of the present invention, the fraction is selected from the group consisting of IE1-2, IE1-3, IE3-2, and IE3-3.

In one example of the invention, the sub-fraction may be obtained by further purification through gel filtration chromatography. In one example, the sub-fraction is IE3-3G1

In the present invention, the G3PDH or the functional variant or fragment thereof can be manufactured into a pharmaceutical composition or food composition with a physiologically acceptable excipient or diluent. In one example, the pharmaceutical composition or food composition may be manufactured from the PM-A0005 strain, or the extract, the fraction, and the sub-fraction thereof.

According to the present invention, the PM-A0005 strain was delivered to the mice with OVA or Der p allergens through an oral administration, and the inflammation of skin was also significantly reduced, demonstrating that the PM-A0005 strain is effectively in treating or preventing atopic dermatitis.

According to the present invention, one skilled in the art of the present invention can select the proper material needed to manufacture the compositions in the form of an oral pharmaceutical (such as a drug), food, or drink composition. The food or drink includes, but is not limited to, dairy product, fermented dairy product, drink, sports drink, nutritional additive, health food, candy, or gum.

The present invention is further illustrated by the following examples, which are provided for the purpose of demonstration rather than limitation.

### Example 1

### Preparation of the PM-A0005 Strain

Lactobacillus gasseri PM-A0005 (PM-A0005 strain) was cultured in MRS medium at 37° C. for 24 hours, and a single colony was selected and cultured in 5 ml sterile medium at 37° C. for 20 hours. On the third day, a seed tank was made in sterile method, the broth taken and diluted by a factor of 50 with the MRS medium, and then incubated at 37° C. for 16 hours. On the fourth day, 100 ml of the broth was inoculated in Microprocessor Control Fermenter (model: Major Science, MS-F1), containing 3.5 liters of the MRS medium to be fermented, and cultured at pH 6.0 at 37° C. for 5.5 hours. After fermentation, the broth was centrifuged at a high speed and the supernatant was removed. The number of colonies in the bacterial pellet was 10⁹.

TABLE 1
Formula of fermentation test medium

| Name | Proportion of added |
|---|---|
| Glucose | 2% |
| Yeast extract | 3% |
| Meat Peptone S2; Number: 19518 | 3% |
| dipotassium phosphate | 0.2% |
| magnesium sulfate | 0.005% |
| manganese sulfate | 0.001% |
| sodium acetate | 0.1% |
| triammonium citrate | 0.05% |
| calcium carbonate | 0.1% |
| Tween 80 | 0.1% |

### Example 2

### Preparation of the Extract of the PM-A0005 Strain

The bacterial pellet of the PM-A0005 strain prepared by Example 1 was washed three times by PBS and homogeneously suspended with 50 mM Tris-HCl (pH 8.0), 1% lysozyme added, and then placed on ice for 2 hours to release its cytoplasmic contents. Next, the reaction was centrifuged at 22,500 g and 4° C. for 30 minutes. After the reaction was centrifuged, the supernatant (cytosolic fraction) and the precipitate (cell-wall fraction) were separated. The supernatant was placed on ice and ammonium sulfate powder was slowly added to reach the needed concentration (0˜25%). After the target concentration was achieved, the solution was stirred for 10 minutes. Next, the solution was centrifuged again at 22,500 g and 4° C. for 30 minutes. The precipitated protein was recovered with appropriated volume of 50 mM Tris-HCl (pH 8.0). Simultaneously, the centrifuged supernatant was precipitated with ammonium sulfate repeatedly in concentrations of 25˜50%, 50˜75% and 75˜100%. The protein extracts in 4 concentrations were obtained: AS_{—}0-25%, AS_{—}25-50%, AS_{—}50-75%, AS_{—}75-100%. The 4 collected protein extracts were placed in 5 L of 50 mM Tris-HCL (pH 8.0) at 4° C. for 24 hours of dialysis on a dialysis membrane (Mw: 6,000˜8,000). After the dialysis, the solution was centrifuged again at 22,500 g and 4° C. for 30 minutes. The supernatant was collected and concentrations of the proteins were measured.

### Example 3

### Isolation of Mice Dendritic Cells and Measurement of Interleukin-12 (IL-12)

The mice were euthanized to obtain their legs. The muscular tissue of the legs was removed, and the cells were washed out with 5-10 ml of medium, injected by syringes. The cell suspension was centrifuged at 4° C. and 1,500 rpm for 10 minutes, and the supernatant was removed. 1 ml red blood cells dissolution buffer was added for 1 minute to remove the red blood cells, 9 ml PBS was subsequently added, and the cell suspension was centrifuged at 4° C. and 1,500 rpm for 10 minutes to precipitate the cells. The supernatant was removed; the cells were washed twice with PBS, and suspended in the medium. IL-4 (1 μl/ml) and GM-CSF (1.5 μl/ml) were added to the co-culture to enhance cells differentiate into dendritic cells. After 8 days of incubation, cells were collected and the cell concentration was adjusted to 4×10⁶/ml. Dendritic cells and the analyte were co-cultured for 48 hours, and the supernatant of the cell incubation was collected. The quantity of IL-12p40 in the supernatant was detected by ELISA.

The four protein extracts (10 μg) were co-cultured with mice dendritic cells, and the secretion amount of cytokine IL-12 (IL-12p40) in mice dendritic cells were measured through ELISA to evaluate the effectiveness of immune regulation. The result showed that AS_{—}0-25%, AS_{—}25-50%, AS_{—}50-75% and AS_{—}75-100% stimulated mice dendritic cells to generate 544.62 pg/ml, 315.90 pg/ml, 597.27 pg/ml, and 159.80 pg/ml IL-12, respectively (Table 2). AS_{—}0-25% and AS_{—}50-75% induced mice dendritic cells to express IL-12 more than 3 times higher than that of the control group (cells only).

TABLE 2
The data of 4 groups of ammonium sulfate-precipitating proteins in stimulating mice dendritic cells to generate IL-12p40.

| | | | | | mouse dentric cell | | | | cell-free | | | |
|---|---|---|---|---|---|---|---|---|---|---|---|---|
| | | Cell | LPS | PHA | AS | AS | AS | AS | AS | AS | AS | AS |
| | medium | only | 10 ug/ml | 30 ug/m; | 0-25% | 25-50% | 50-75% | 75-100% | 0-25% | 25-50% | 50-75% | 75-100% |
| 1st | 0.00 | 126.67 | 432.31 | 466.16 | 529.23 | 342.57 | 584.10 | 173.08 | −0.25 | −0.51 | 0.26 | 0.26 |
| 2nd | 1.28 | 120.52 | 506.41 | 545.90 | 493.08 | 300.26 | 615.39 | 154.87 | 0.26 | 0.26 | 1.03 | 0.00 |
| 3rd | −1.02 | 111.28 | 466.67 | 532.05 | 611.54 | 304.87 | 592.31 | 151.54 | 0.00 | 0.52 | −0.51 | −0.77 |
| Avg. | 0.09 | 119.49 | 468.46 | 514.70 | 544.62 | 315.90 | 597.27 | 159.83 | 0.00 | 0.09 | 0.26 | −0.17 |
| stdev | 1.16 | 7.74 | 37.08 | 42.61 | 60.71 | 23.21 | 16.22 | 11.59 | 0.26 | 0.53 | 0.77 | 0.53 |

### Example 4

### Preparation of the Fraction of the PM-A0005 Strain

The fractions of the ammonium sulfate-precipitated protein obtained from Example 2 were preliminary isolated from the ion-exchange chromatography system (BioLogic Duo Flow Chromatography System, Bio-Rad). The ion-exchange chromatography column was DEAE-FF (HiTrap™, GE Healthcare). Before the protein chromatography was done, the ion-exchange column balanced with Buffer A (50 mM Tris-HCl, pH 8.5), using more than twice the amount of the column volume. The protein sample was subsequently poured in, purified with Buffer A, using more than 6 times the column volume; simultaneously, the partition collector was started in order to collect the effluent, 1 ml per column. After collecting 30 ml, Buffer B (50 mM Tris-HCl and 1M NaCl, pH 8.5) was used to elute the protein through a salinity gradient. Finally, the residual protein was washed out by 100% Buffer B. Three fractions, IE1-1, IE1-2, and IE1-3, were isolated from AS_{—}0-25%; and 3 fractions, IE3-1, IE3-2 and IE3-3, were isolated from AS_{—}50-75%.

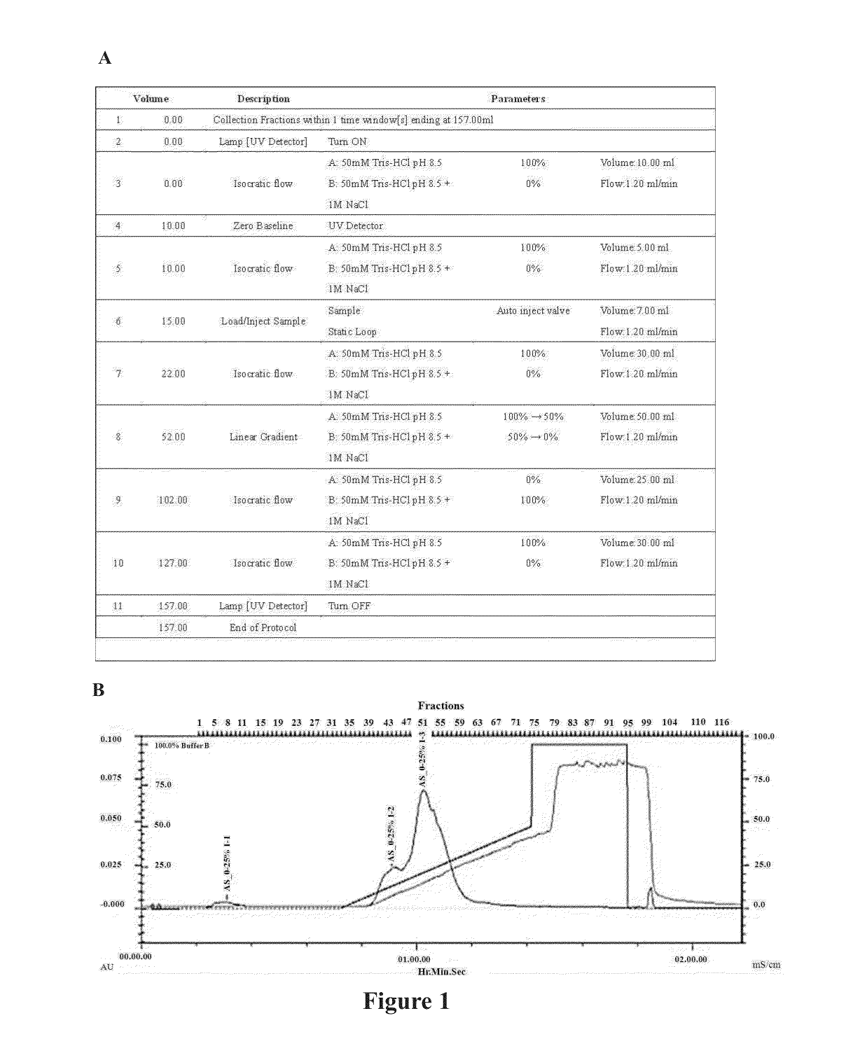

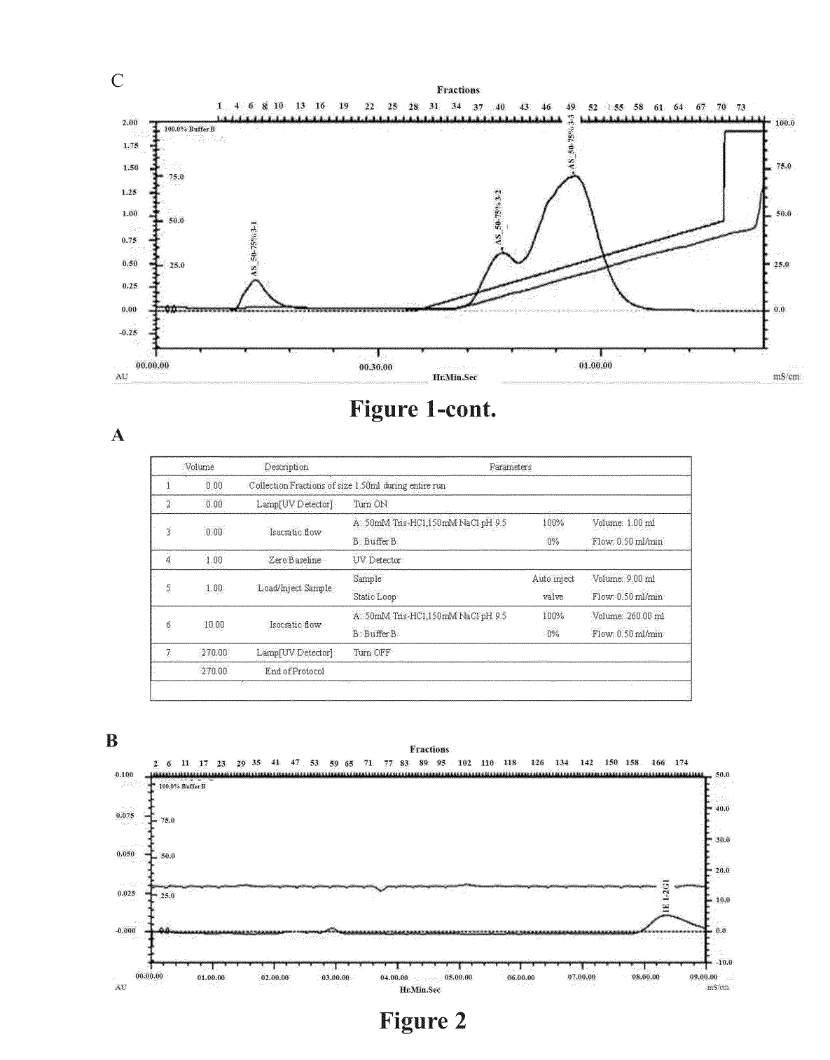

The fractions isolated from AS_{—}0-25% and AS_{—}50-75% were further isolated through ion-exchange chromatography (FIG. 1A). Through ion-exchange chromatography, 3 fractions, IE1-1, IE1-2 and IE1-3, were isolated from AS_{—}0-25% (FIG. 1B); and 3 fractions, IE3-1, IE3-2 and IE3-3, were isolated from AS_{—}50-75% (FIG. 1C).

The effectiveness of immune regulation of the six fractions was evaluated by measuring the secretion amount of cytokine IL-12 (IL-12p40) in mice dendritic cells. The result demonstrated that IE1-1, IE1-2, IE1-3, IE3-1, IE3-2, and IE3-3 did stimulate mice dendritic cells to generate IL-12, 427.6 pg/ml, 1135.85 pg/ml, 1213.2 pg/ml, 258.87 pg/ml, 1098.41 pg/ml, and 1850.39 pg/ml, respectively (Table 3); wherein IE1-2, IE1-3, IE3-2, and IE3-3 induced the mice dendritic cells to express IL-12 more than 3 times the amount of the control group (cells only).

TABLE 3
The data of 6 groups of ion-exchange isolating samples in
stimulating mice dendritic cells to generate IL-12p40.

| | | | LPS | PHA | |
|---|---|---|---|---|---|
| | medium | cell | 10 ug/ml | 30 ug/ml | IE1-1 |
| 1st | −0.38 | 169.62 | 1796.54 | 1588.08 | 201.93 |
| 2nd | −1.15 | 164.23 | 1689.62 | 1678.85 | 191.93 |
| 3rd | 0.00 | 169.65 | 713.10 | 703.44 | 679.31 |
| 4th | 0.00 | 156.55 | 889.65 | 737.24 | 637.24 |
| Avg. | −0.39 | 165.01 | 1272.23 | 1176.90 | 427.60 |
| STDEV | 0.54 | 6.19 | 550.18 | 528.67 | 266.94 |
| | IE1-2 | IE1-3 | IE3-1 | IE3-2 | IE3-3 |
| 1st | 1522.69 | 1508.08 | 248.85 | 1722.69 | 1878.85 |
| 2nd | 1563.46 | 1668.85 | 274.23 | 1775.77 | 1821.93 |
| 3rd | 696.55 | 717.93 | 257.24 | 447.58 | 1850.39 |
| 4th | 760.69 | 957.93 | 255.17 | 447.58 | 1850.39 |
| Avg. | 1135.85 | 1213.20 | 258.87 | 1098.41 | 1850.39 |
| STDEV | 471.25 | 449.08 | 10.84 | 751.82 | 23.24 |

### Example 5

### Preparation of the Sub-Fraction of the PM-A0005 Strain and Measurement of Activity

The fractions, IE1-2, IE1-3, IE3-2, and IE3-3, obtained from the isolation with ion-exchange chromatography in Example 4, were further isolated with gel-filtration chromatography (Sephacryl™ S-300 HR Column, Amersham Bioscience). Before progressing, the column and the operated machine were balanced with the buffer (50 mM Tris-HCl, pH 9.5, 50 mM NaCl) in over twice the column volume. The sample was injected into the column with a syringe, with the sample volume being less than 3% of the gel volume. After the sample was injected, the progress proceeded with predetermined flow rate of 0.5/min. Finally, the sample was collected and analyzed using protein quantitation and activity analysis.

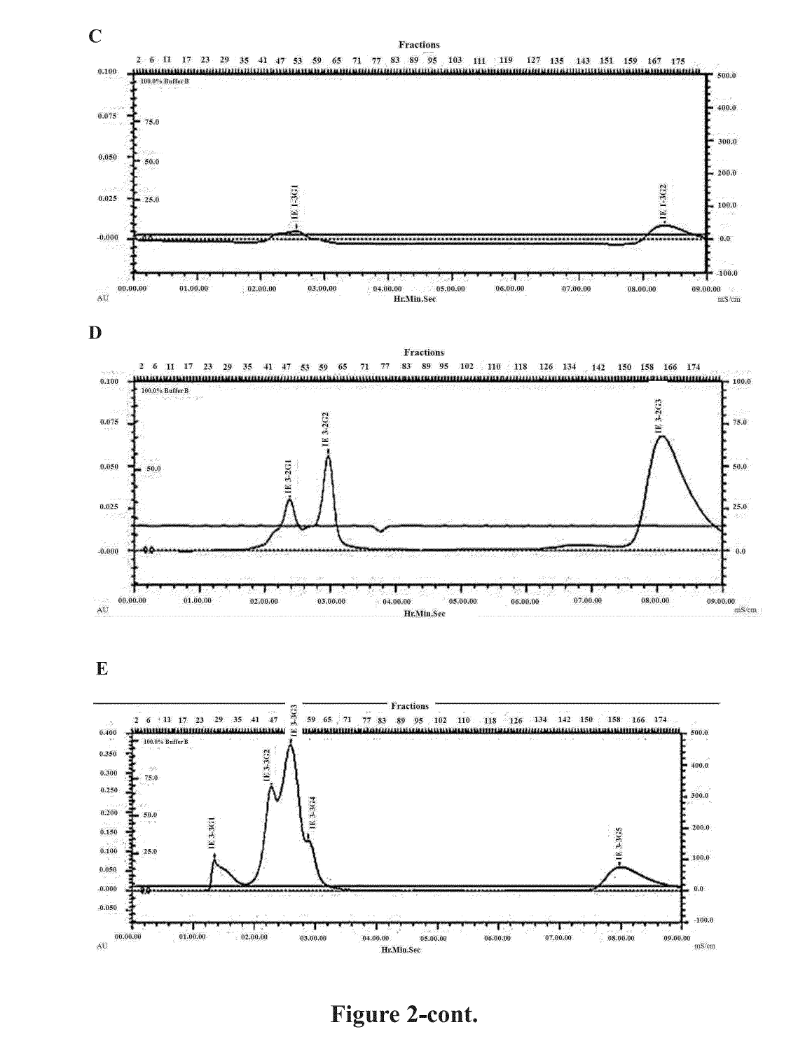

The fractions, IE1-2, IE1-3, IE3-2, and IE3-3, were further isolated with gel-filtration chromatography to isolate the active ingredient (FIG. 2A). Through gel-filtration chromatography, the sub-fraction, IE1-2G1, was isolated from IE1-2 (FIG. 2B); 2 sub-fractions, IE1-3G1 and IE1-3G2, were isolated from IE1-3 (FIG. 2C); 3 sub-fractions, IE3-2G1, IE3-2G2, and IE3-2G3, were isolated from IE3-2 (FIG. 2D); 5 sub-fractions, IE3-3G1, IE3-3G2, IE3-3G3, IE3-3G4, and IE3-3G5, were isolated from IE3-3 (FIG. 2E).

The effectiveness of immune regulation of the 11 sub-fractions, obtained by the isolation with gel-filtration chromatography, was evaluated by measuring the secretion amount of cytokine IL-12 (IL-12p40) in mice dendritic cells. The result demonstrated that IE1-2G1, IE1-3G1, IE1-3G2, IE3-2G1, IE3-2G2, IE3-2G3, IE3-3G1, IE3-3G2, IE3-3G3, IE3-3G4, and IE3-3G5 stimulated mice dendritic cells to generate 176.5 pg/ml, 559.63 pg/ml, 327.75 pg/ml, 813.38 pg/ml, 398.75 pg/ml, 234.50 pg/ml, 1493.63 pg/ml, 215.13 pg/ml, 203.63 pg/ml, 227.38 pg/ml, and 245.50 pg/ml IL-12, respectively (Table 4). IE3-3G1 induced the mice dendritic cells to express IL-12 more than 7.3 times more than the control group (cells only).

TABLE 4
The data of 11 groups of gel-filtration isolating samples
in stimulating mice dendritic cells to generate IL-12p40.

| | | | LPS | PHA | | | | | |
|---|---|---|---|---|---|---|---|---|---|
| | Medium | Cell only | 10 ug/ml | 30 ug/ml | IE1-2G1 | IE1-3G1 | IE1-3G2 | IE3-2G1 | IE3-2G2 |
| 1st | 1.25 | 270.75 | 1369.75 | 1411.75 | 183.00 | 920.75 | 487.00 | 448.25 | 393.00 |
| 2nd | 0.50 | 136.50 | 1743.50 | 1813.00 | 170.00 | 198.50 | 168.50 | 1178.50 | 404.50 |
| Avg. | 0.87 | 203.63 | 1556.63 | 1612.38 | 176.50 | 559.63 | 327.75 | 813.38 | 398.75 |
| Stdev | 0.53 | 94.93 | 264.28 | 283.73 | 9.19 | 510.71 | 225.21 | 516.36 | 8.13 |

| | IE3-2G3 | IE3-3G1 | IE3-3G2 | IE3-3G3 | IE3-3G4 | IE3-3G5 |
|---|---|---|---|---|---|---|
| 1^{st} | 258.50 | 1325.75 | 259.25 | 259.75 | 275.75 | 294.00 |
| 2^{nd} | 210.50 | 1661.50 | 171.00 | 147.50 | 179.00 | 197.00 |
| Avg. | 234.50 | 1493.63 | 215.13 | 203.63 | 227.38 | 245.50 |
| stdev | 33.94 | 237.41 | 62.40 | 79.37 | 68.41 | 68.59 |

### Example 6

### Active Ingredient of the PM-A0005 Strain Reduced Allergic Asthma in Mice

Material and Method

1. Preparation of Animal Model with Mice in Der p Allergy

The Der p extract was well-mixed with aluminum hydroxide, and allergic mice were intraperitoneally injected in one week intervals, with a total of 3 injections. One week after the 3^{rd} injection, allergens were inoculated in the trachea to induce a response. The mice were anaesthetized after weighing, the tongue was clipped with forceps, and 50 μl Der p antigen solution (0.5 mg/ml) was dropped in the throat with a microsyringe. The clip was released after the mice made a choking noise, the tongue unhitched, and the mice illuminated with a light, to help them wake up, until the mice were able to maintain an upright position for 1 minute.

2. Collection of Bronchioalveolar Irrigant and Leukocyte Classification

1 ml of warm sterile physiological saline was slowly injected into the lung by a needle to fully expand and wash the lung, and then withdrawn. After repeating this step, the sterile physiological saline, irrigant, was placed in the ice for temporary storage. The bronchioalveolar irrigant, about 1.8 mL, which was removed from the lungs after they were washed twice was centrifuged at 1,200 rpm at 4° C. for 10 minutes. The supernatant was collected and stored in a −70° C. refrigerator. The precipitated cells were stained with eosin Y, and the cell number calculated. 50-100 μl bronchioalveolar irrigant was taken, and a smear was made with a cytospin centrifuge, the stain was observed under an immersion lens, and the number of leukocyte was calculated.

3. Tissue Sections Staining

First, the fixed tissue sections were dewaxed and rehydrated. After the tissues were immersed in distilled water, reacted with 0.5% periodic acid for 15 minutes, and then washed under running water for 5 minutes. Next, the fixed tissue sections re-immersed in distilled water, and reacted with Schiff's reagent for 5 minutes. Finally, the sections were washed under running water for 5 minutes, and then dehydrated and sealed. The tissue sections were observed under light microscopy; the glycoprotein and the neutral mucus were red, and the nucleus was blue.

4. Measurement of Airway Resistance in Mice

Small animal unrestrained whole body plethysmography (BUXCO) was used to measure airway resistance (enhance pulsed). 0 mg/ml, 6.25 mg/ml, 12.5 mg/ml, 25 mg/ml, and 50 mg/ml of methacholine were dissolved in PBS, and were administered to the mice by a spray. The airway resistance value (herein Penh value) was measured as evaluation standard.

5. Isolation of Mice Spleen Cells and Measurement of IFN-γ

Mice spleen mixed with an appropriate amount of PBS. The residual tissue and spleen cells were isolated at 500 rpm centrifugation, the supernatant, which contained spleen cells, was isolated with Ficoll (herein isolated at 16° C. and 720 g for 25 minutes), and then wash with PBS 3 times. The cells were suspended in RPMI-1640 medium, cell number calculated, and the concentration was adjusted to 4×10⁶/ml. The spleen cells and the analyte were co-cultured for 48 hours, and the supernatant of cell incubation was collected. The quantity of IFN-γ in the supernatant was detected by ELISA.

Results

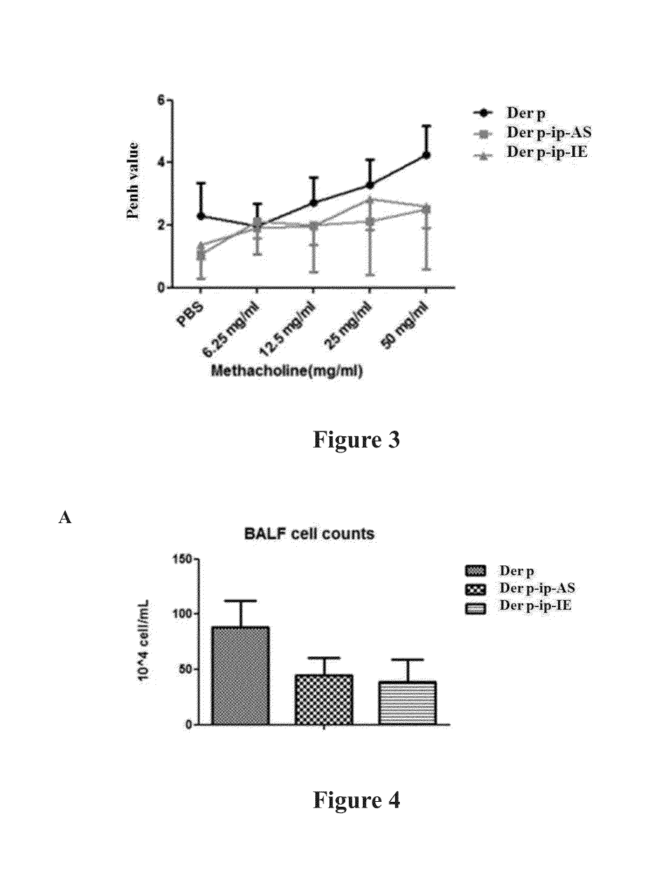

Der p allergen-sensitized mice were used to evaluate whether the extract AS_{—}50-75% (AS) and the sub-fraction IE3-3G1 (IE) were effective in reducing an allergic response. Airway hyperresponse (AHR) would be initiated after Der p induced an airway allergic asthma response in mice. Here, the resistance of mice airway against the bronchoconstriction agent, methacholine, was measured using a non-invasive method. The lung function of mice was evaluated by an airway resistance value (herein Penh value); the higher Penh value represents larger mice airway resistance. As shown in FIG. 3, in the positive control group (Der p), when the concentration of methacholine was increased, the Penh valued increased as well. However, when the groups that were given the extract AS_{—}50-75% (Der p allergy-AS) and the sub-fraction IE3-3G1 (Der p allergy-IE), the Penh values decreased significantly. Thus, the extract AS_{—}50-75% (Der p allergy-AS) and the sub-fraction IE3-3G1 (Der p allergy-IE) reduced airway resistance, and eased an asthma attack.

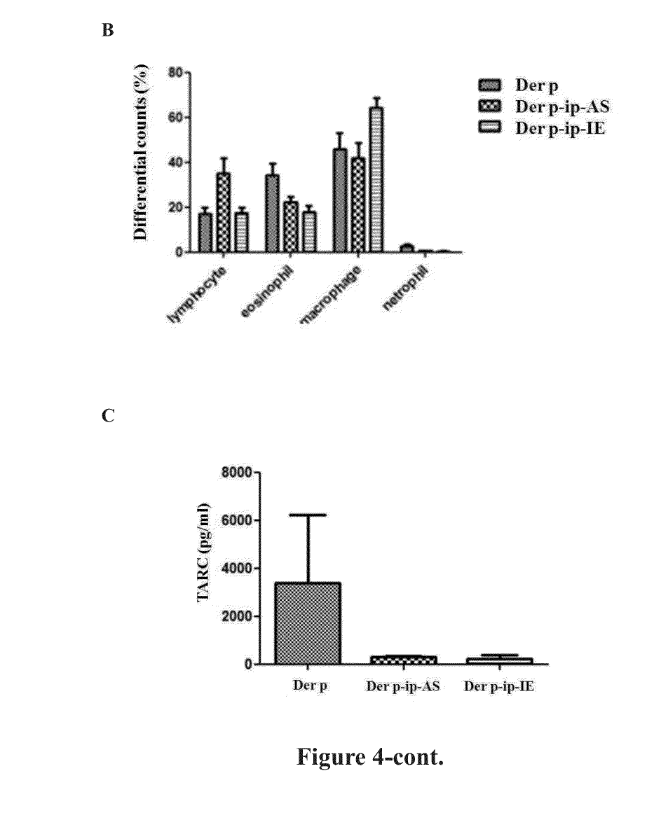

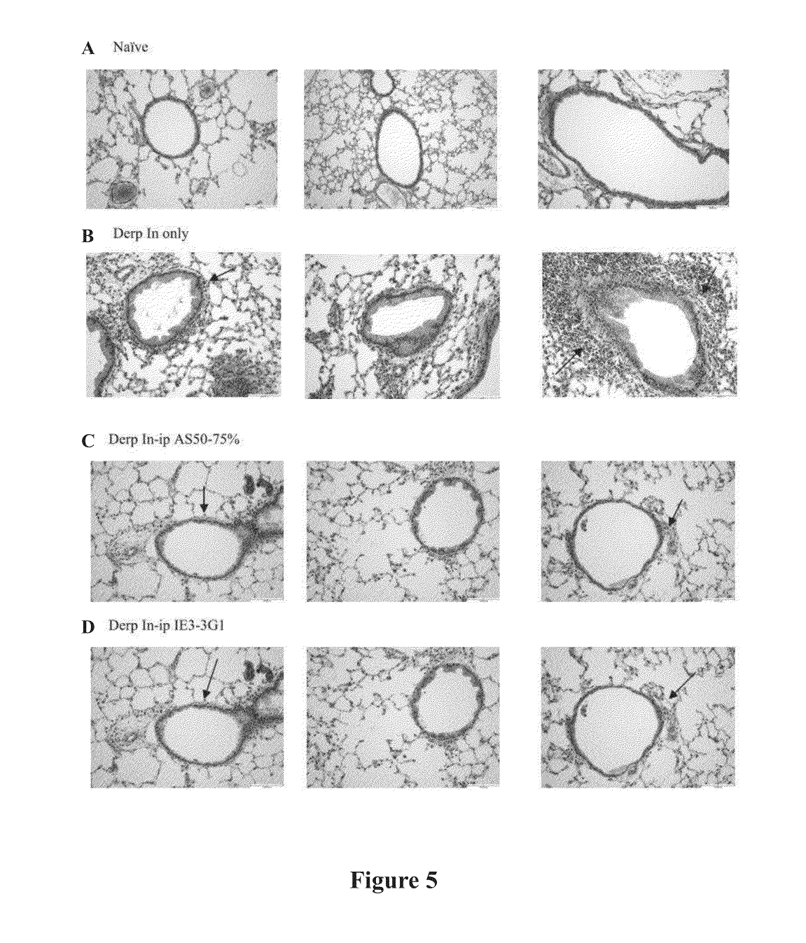

In addition to airway resistance, the lung cell irrigant was also studied. The lung cell irrigant of Der p allergen-sensitized mice had a significant increase in cell number (FIG. 4A), showing there was inflammation and cell infiltration in the lungs. The administration of the extract AS_{—}50-75% and the sub-fraction IE3-3G1 significantly decreased the number of cells in the lung cell irrigant, and is effective in inhibiting inflammation, as seen from the significant decrease in the proportion of eosinophils and neutrophils (FIG. 4B). The concentration of thymus and activation-regulated chemokine (TARC) in lung cell irrigant was also measured. In mice administered with the extract AS_{—}50-75% and the sub-fraction IE3-3G1, the concentration of TARC decreased significantly when compared to the positive control (FIG. 4C). Therefore, the administration of the extract AS_{—}50-75% and the sub-fraction IE3-3G1 reduced the thickening of the airway wall and decreased the number of immune cells infiltration in mice, which can be observed through H&E staining of alveolar tissue sections (FIG. 5).

It was demonstrated by the result that the extract AS_{—}50-75% and the sub-fraction IE3-3G1 effectively decreased Der p allergen-sensitized mice airway resistance and significantly inhibited cells infiltration and inflammation in lung cells, showing that the mice airway allergic response were effectively reduced and asthma attacks were eased due to the administration of the extract and sub-fraction of the PM-A0005 strain the invention.

### Example 7

### Active Ingredient of the PM-A0005 Strain and the PM-A005 Strain Itself Reduced Atopic Dermatitis in Mice

Material and Method

1. Preparation of Animal Model with Mice in Der p or Ovalbumin (OVA) Allergy

The animal model with mice in percutaneous sensitization:

The mice were anaesthetized and fixed the 1*1 cm² gauze on neck back of mice, wherein the gauze was mixed with 100 μl PBS solution (137 mM NaCl, 2.7 mM KCl, 10 mM Na₂HPO₄, 1.8 mM KH₂PO₄, pH 7.2, dissolved in 1 L ddH₂O) and 50 μg Der p extract per mouse or 100 μg OVA per mouse. After seven days, removed the gauze and let the mice rest for fourteen days. The above steps of percutaneous sensitization were repeated twice, and sacrificed the mice at the fiftieth day. The percutaneous sensitization of the PM-A0005 strain administration model was the same as above steps, and administered 200 μl PBS solution, 1*10⁷ CFU or 1*10⁹ CFU of the PM-A0005 strain to the mice. The mice were sacrificed at the fiftieth day. The percutaneous sensitization of the sub-faction IE3-3G1 intraperitoneal injection model was the same as above steps, and intraperitoneal injected PBS solution, or 25 μg the sub-fraction IE3-3G1 when fixed on the gauze (three times). The mice were sacrificed at the fiftieth day.

2. Measurements of Skin Physiological Condition

The mice were anaesthetized, and the moisture content and pH value of skin were measured by Cutometer® MPA 580. The trans-epidermal water loss (TEWL) of skin was measured by Tewameter TM210.

3. Cell Proliferation and Cytokine Secretion of Mice Spleen Cells

The spleens of mice were taken out, and ground it. The spleen cells were isolated at 5 min centrifugation (300 g, 4° C.). The supernatant was removed, and the spleen cells were dissolved with 3 ml RBC (red blood cell) lysing buffer. The lysing reaction was stopped by adding 3 ml cRPMI (RPMI-1640 medium supplemented with 2 mM L-glutamine, 50 mM 2-mercaptoethanol (2-ME), 1 mM sodium pyruvate, 0.5% Penicillin-Streptomycin, 1 mM non-essential amino acids, and Fetal Bovine Serum, pH 7.2). The supernatant was removed, and the spleen cells were suspended with 1 ml cRPMI. The cell numbers were calculated, and the concentration was adjusted to 4×10⁶/ml.

The Der p [symbol] OVA or leucoagglutinin (PHA-L) were added in spleen cells, and co-cultured for 48 and 72 hrs. The cholecystokinin-8 (CCK-8) was added at the 44 and 68 hr. The cell proliferation of spleen cell was measured by detecting the absorbance on wave length 450 nm. Besides, the spleen cells and the Der p [symbol] OVA or PHA-L were co-cultured for 48 hours, and the supernatant of cell incubation was collected. The quantities of IFN-γ, IL-10, and IL-17 in the supernatant were detected by ELISA.

4. Flow Cytometer Analysis of Mice Lymphocytes Grouping

The axillary lymph nodes were taken out, and the cells were suspended with RPMI containing 2% fetal bovine serum. The cell numbers were adjusted to 5×10⁵/tube, and the cells were isolated at 5 min centrifugation (400 g, 4° C.), and the supernatant was removed. The CD3 [symbol] CD4 [symbol] CD25 antibodies were added in the cells, and resuspended with 1 ml RPMI containing 2% fetal bovine serum. The cells were isolated at 5 min centrifugation (400 g, 4° C.), and treated with Fixation/Permeabilization Buffer.

5. Measurements of Total Immunoglobulin E (IgE) Antibody, and OVA and Der p Specific IgE Amount.

The IgE capture antibody, OVA, and Der p were separately diluted with 50 mM carbonate coating buffer, and added in 96 wells plate with 100 ml per well. The 96 wells plate was stood at room temperature for 1 hr, and washed by TBST (50 mM Tris-base, 0.14M NaCl and 0.05% Tween 20 in 1 L ddH₂O, pH 8.0). Each 96 wells plate was added in 200 ml blocking buffer (50 mM Tris-base, 0.14M NaCl, and 1% BSA in 1 L ddH₂O, pH 8.0), and stood at room temperature. The 96 wells plate was washed by TBST. Each 96 wells plate was added in 100 ml mice serum or standard IgE of mice that diluted with sample/conjugate diluents. The 96 wells plate was stood at room temperature and washed by TBST. The 96 wells plate was added in HRP-conjugated IgE detection antibody, and stood at room temperature without light. The 96 wells plated was washed by TBST, and added in the 3,3′5,5′-tetramethyl benzidine (TMB) to pigmented. Finally, the 96 wells plate was added in 2N H₂SO₄ to terminate the pigment reaction, and measured the absorbance with wave length in 450 nm.

6. Tissue Sections Staining

The fixed tissue sections were dewaxed and rehydrated, and conducted with immunohistochemistry stain and hematoxylin-eosin stain (H&E stain).

A. Immunohistochemistry Stain

The slides were rehydrated and put in sodium citrate buffer (10 mM sodium citrate, 0.05% Tween20, pH 6, dissolved in 1 L ddH₂O), and stood at room temperature for 5 min with high pressure. The tissues on slides were circled by PAP pen, and the slides were soaked in TBST. The slides were dropped with peroxidase blocking agent, and washed by TBST. Then the slides were dropped with protein blocking agent, and washed by TBST. The antibodies of thymic stromal lymphopoietin (TSLP), [symbol] Interleukin-17 and langerin were dilutes with normal antibody diluent. The slides were washed by TBST, and dropped in the NovoLink™ Polymer and stood for 10 min. The slides were washed by TBST, and dropped in the 3,3′-diaminobenzidine (DAB) for pigmentation. The reaction was stopped by adding ddH₂O. The hematoxylin was added in the slides, and stopped the reaction by adding ddH₂O. The slides were dehydrated and then sealed.

B. Hematoxylin and Eosin Stain (H&E Stain)

The slides were stained with hematoxylin for 5 min, and washed under running water and re-immersed in distilled water. Then, the slides were stained with eosin for 30 s, and immersed in ethanol and xylene. The slides were dehydrated and then sealed.

Results

1. The PM-A0005 Strain or the Sub-Fraction IE3-3G1 can Rescue the Physiological Features of Atopic Dermatitis

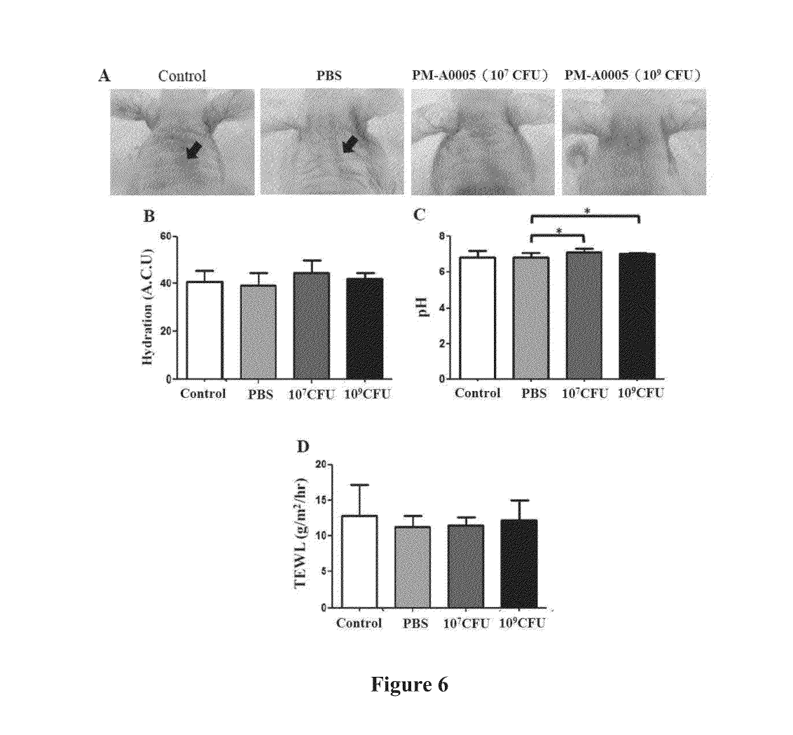

The groups of oral treatment with 1×10⁷ CFU or 1×10⁹ CFU of the PM-A0005 strain had smoother skin and produce less dander than the groups of control and PBS (FIG. 6A). The hydration of skin between different groups did not have significant difference (FIG. 6B). The groups of oral treatment with 1×10⁷ CFU or 1×10⁹ CFU of the PM-A0005 strain had higher pH values (FIG. 6C). The TEWL of skin between different groups did not have significant difference (FIG. 6D).

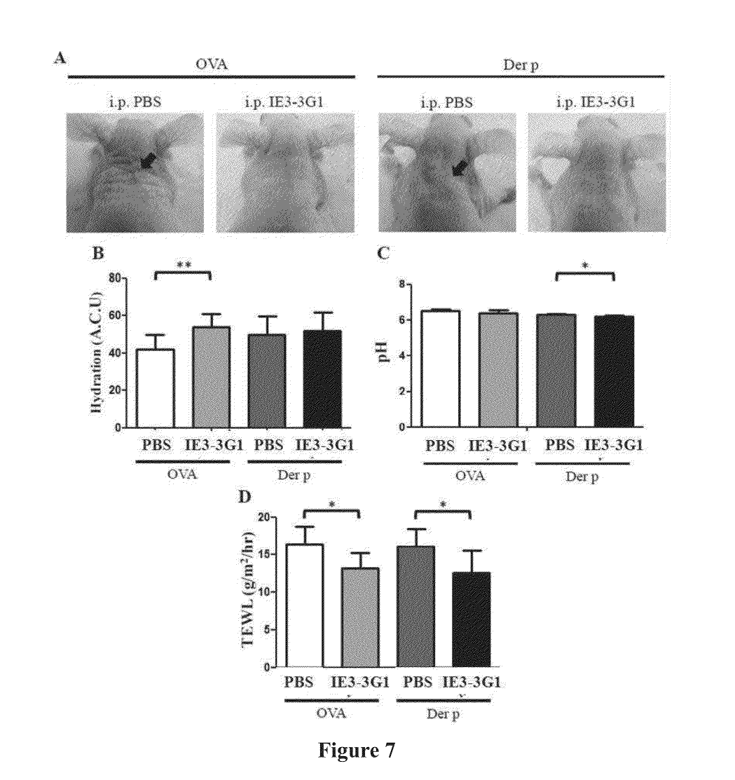

The groups of OVA or Der p percutaneous sensitization followed by intraperitoneal injection with IE3-3G1 had smoother skin, less wrinkle of skin, and produce less dander than the groups of PBS (FIG. 7A). The groups of intraperitoneal injection with IE3-3G1 had higher hydration of skin than the group of PBS, wherein the group of OVA percutaneous sensitization had the significant difference (FIG. 7B). The groups with Der p percutaneous sensitization followed by intraperitoneal injection with IE3-3G1 had less pH value of skin than the group of PBS (FIG. 7C). Both the groups of OVA or Der p percutaneous sensitization followed by intraperitoneal injection with IE3-3G1 had significantly decreased TEWL than the group of PBS (FIG. 7D).

2. The PM-A0005 Strain or the Sub-Fraction IE3-3G1 Ameliorated the Skin Inflammation of Mice with Atopic Dermatitis

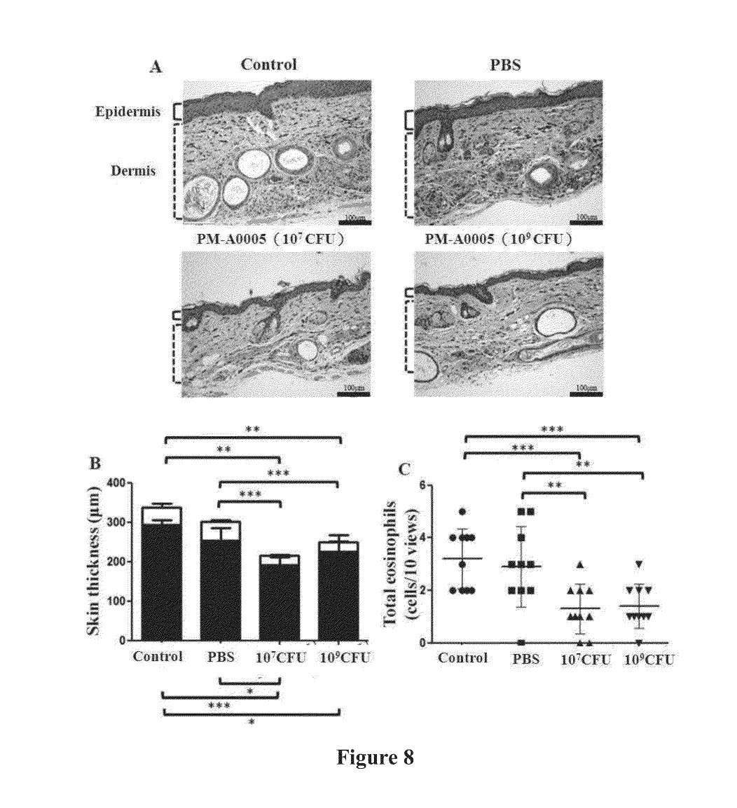

In the H&E stain of skin tissue sections, the epidermal layer and dermis layer of PBS group or control were thickening, and produced large amounts of immune cell infiltration and eosinophils (FIGS. 8A-8C). In the groups of oral treatment with 1×10⁷ CFU or 1×10⁹ CFU of the PM-A0005 strain, the thickening epidermal layer and dermis layer were ameliorated, and the amounts of immune cell infiltration and eosinophils were decreased (FIGS. 8A-8C).

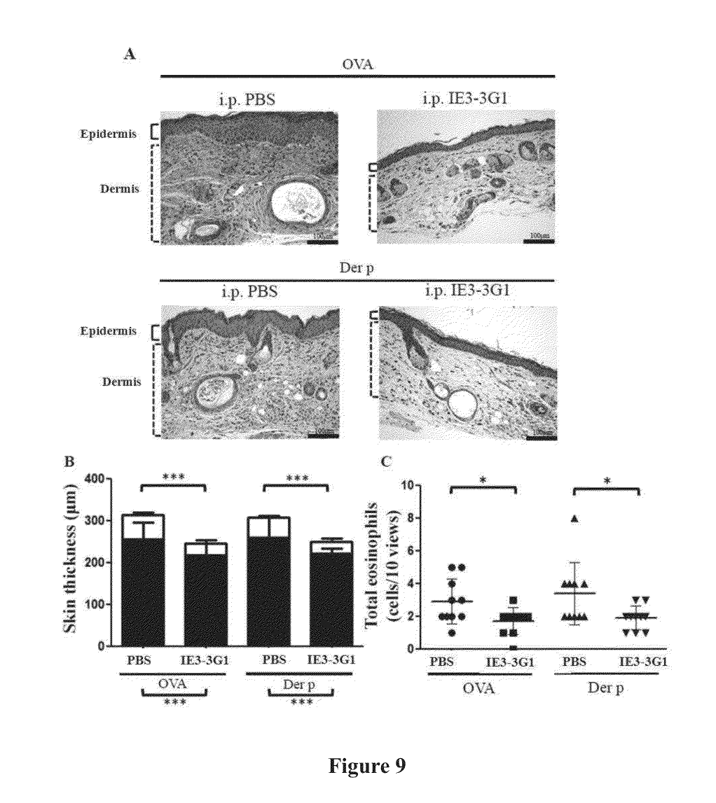

In the groups of OVA or Der p percutaneous sensitization followed by intraperitoneal injection with PBS, the epidermal layer and dermis layer were apparently thickening, and produced large amounts of immune cell infiltration and eosinophils (FIGS. 9A-9C). In the groups of OVA or Der p percutaneous sensitization followed by intraperitoneal injection with the sub-fraction IE3-3G1, the thickening epidermal layer and dermis layer were ameliorated, and the amounts of immune cell infiltration and eosinophils were decreased (FIGS. 9A-9C).

3. The PM-A0005 Strain or the Sub-Fraction IE3-3G1 Decreased the Production of TSLP in Epidermal Layer and Decrease the Translocation of Langerhans' Cells

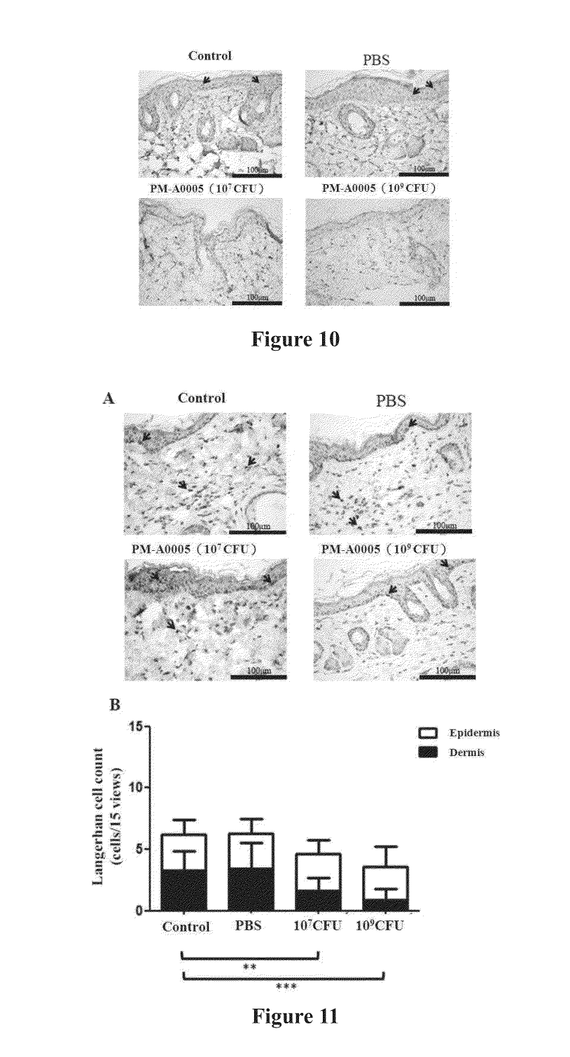

In the results of IHC stain, the groups of control or oral treatment with PBS had the high expression of TSLP in epidermal layer (FIG. 10), and the amount of Langerhans' cells in dermis layer was apparently increased (FIGS. 11A and 11B). In the groups of oral treatment with 1×10⁷ CFU or 1×10⁹ CFU of the PM-A0005 strain, the expression of TSLP in epidermal layer were apparently decreased (FIG. 10), and the amounts of Langerhans' cells in dermis layer were apparently decreased (FIGS. 11A and 11B).

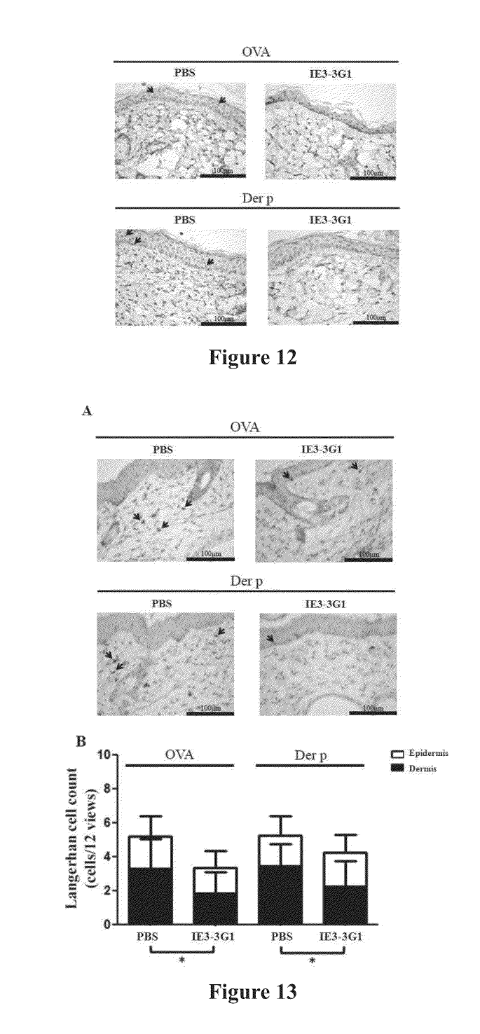

In the groups of OVA or Der p percutaneous sensitization followed by intraperitoneal injection with PBS had the high expression of TSLP in epidermal layer; and the groups of OVA or Der p percutaneous sensitization followed by intraperitoneal injection with IE3-3G1 which the expression of TSLP in epidermal layer were apparently decreased (FIG. 12). In the groups of intraperitoneal injection with PBS, the amount of Langerhans' cells in dermis layer was apparently increased, and the groups of intraperitoneal injection with IE3-3G1 which the amounts of Langerhans' cells in dermis layer were apparently decreased (FIGS. 13A and 13B).

4. The Expression of Total IgE and Antigen-Specific IgE Affect by the PM-A0005 Strain or the Sub-Fraction IE3-3G1

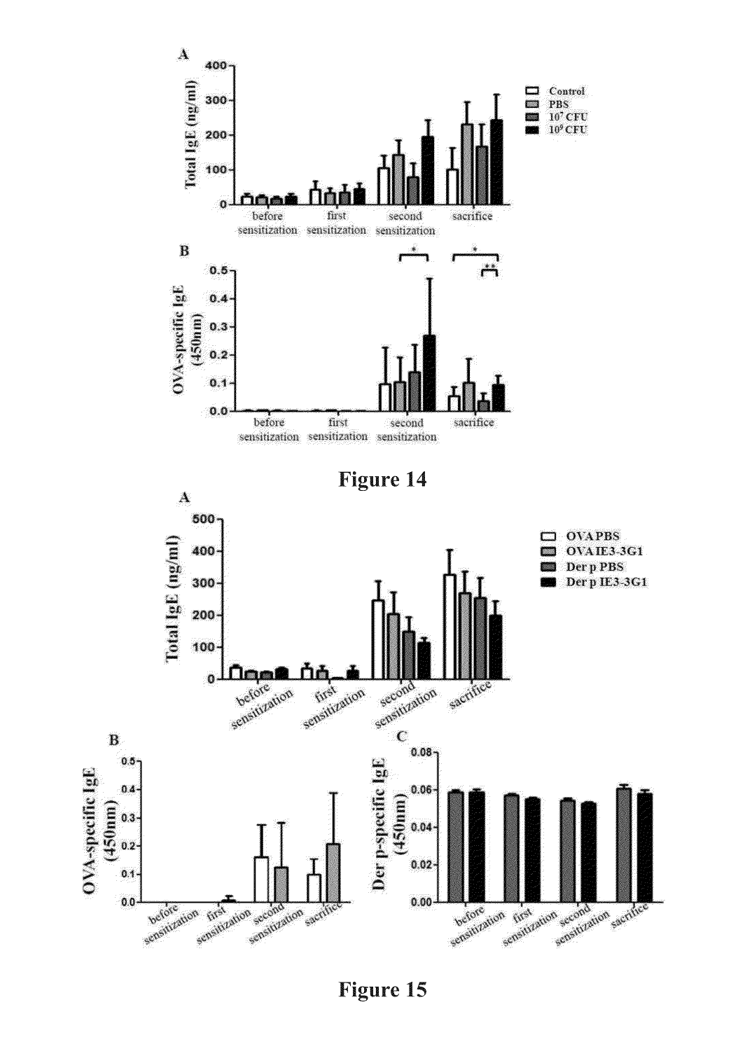

The serums were collected before sensitization, first sensitization, second sensitization, and sacrifice, and the IgE levels were determined by ELISA. The results showed that the total IgE between four groups did not have significant difference (FIG. 14A). The group of oral treatment with 1×10⁷ CFU of the PM-A0005 strain which the OVA specific IgE was apparently decreased than the group of control or oral treatment with 1×10⁹ CFU of the PM-A0005 strain (FIG. 14B).

In the groups of OVA or Der p percutaneous sensitization followed by intraperitoneal injection with IE3-3G1, the total IgE, OVA specific IgE, and Der p specific IgE between four groups did not have significant difference (FIGS. 15A-C).

5. The Proliferation of Spleen Cells Affected by the PM-A0005 Strain or the Sub-Fraction IE3-3G1

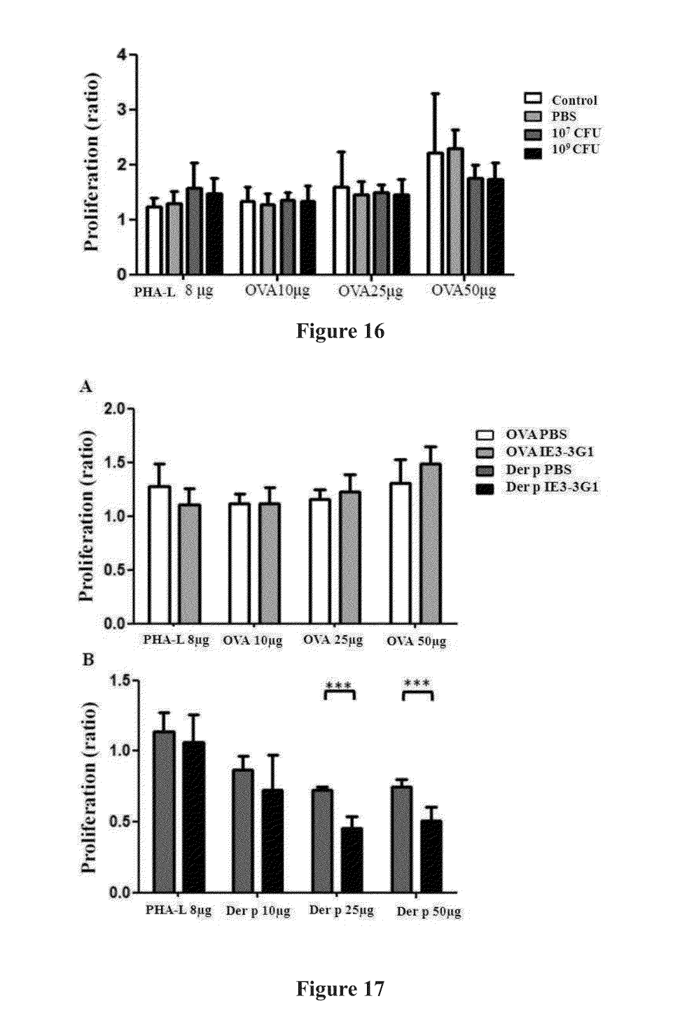

The different concentrations of PHA-L and OVA were added in spleen cells, and the absorbance was measured after 72 hrs. The results show that the proliferation of spleen cells in different concentrations of PHA-L and OVA did not have significant difference between four groups (FIG. 16).

Besides, the different concentrations of PHA-L, OVA and Der p were added in spleen cells, and the absorbance was measured after 72 hrs. In the groups of OVA percutaneous sensitization followed by intraperitoneal injection with IE3-3G1 or PBS, the different concentrations of PHA-L and OVA did not affect the proliferation of spleen cells (FIG. 17A). In the group of Der p percutaneous sensitization followed by intraperitoneal injection with IE3-3G1 had apparently decreased in Der p 25 μg and 50 μg (FIG. 17B).

6. The PM-A0005 Strain or the Sub-Fraction IE3-3G1 Affected the Secretions of Cytokines in Spleen Cells

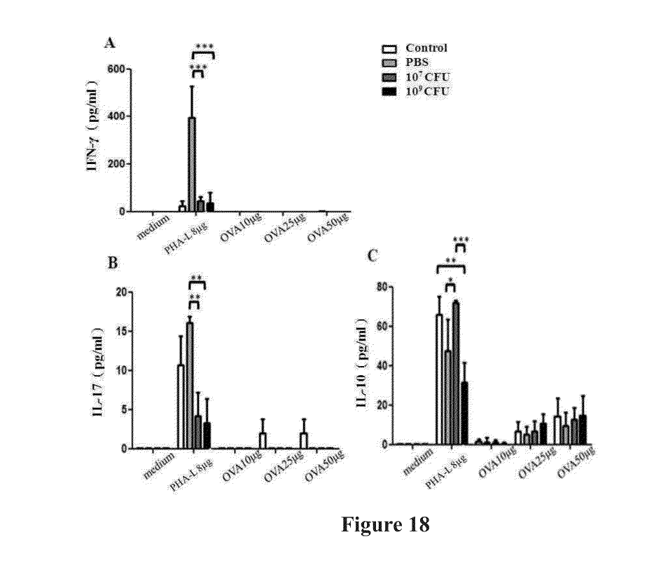

After the administration of PHA-L, the groups of oral treatment with 1×10⁷ CFU or 1×10⁹ CFU of the PM-A0005 strain had decreased the secretion of IFN-γ (FIG. 18A) and IL-17 (FIG. 18B). Besides, the groups of oral treatment with 1×10⁷ CFU or 1×10⁹ CFU of the PM-A0005 strain had increased the secretion of IL-10, wherein the group of 1×10⁷ CFU of the PM-A0005 strain had higher secretion of IL-10 than the group of 1×10⁷ CFU of the PM-A0005 strain (FIG. 18C).

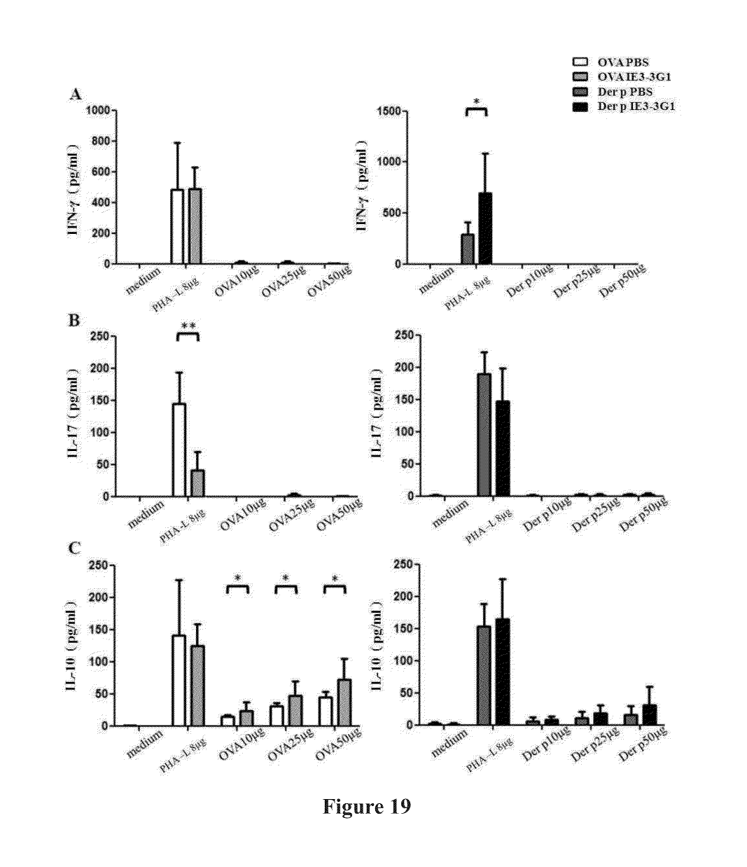

After the administration of PHA-L, the group of Der p percutaneous sensitization followed by intraperitoneal injection with IE3-3G1 had apparently increased the secretion of IFN-γ (FIG. 19A), and the groups of OVA percutaneous sensitization followed by intraperitoneal injection with IE3-3G1 had apparently decreased the secretion of IL-17 (FIG. 19B). Besides, the groups of OVA percutaneous sensitization followed by intraperitoneal injection with IE3-3G1 had apparently increased the secretion of IL-10 when the different concentrations of OVA were administered (FIG. 19C).

7. The PM-A0005 Strain or the Sub-Fraction IE3-3G1 Decreased the Secretions of IL-17 in Skin

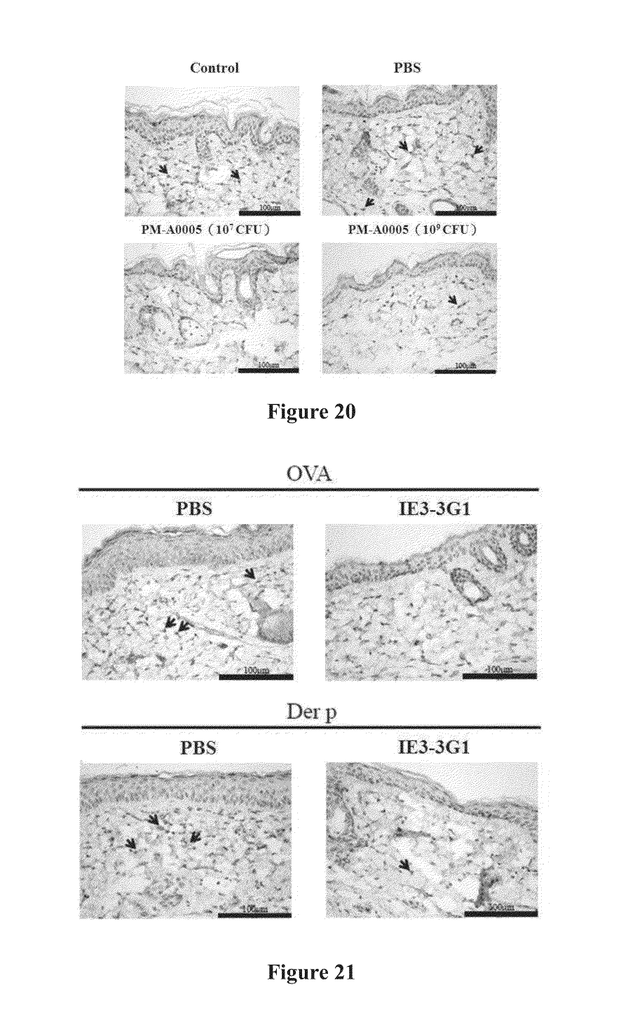

It was shown that the IHC stain, the groups of oral treatment with 1×10⁷ CFU or 1×10⁹ CFU of the PM-A0005 strain had apparently decreased the expression of IL-17 (FIG. 20). In the groups of OVA or Der p percutaneous sensitization followed by intraperitoneal injection with IE3-3G1, the expression of IL-17 was apparently decreased (FIG. 21).

### Example 8

### Identification of the Active Ingredient in IE3-3G1

With two-dimensional electrophoresis analysis, the sub-fraction IE3-3G1 isolated by gel-filtration chromatography was characterized and identified by LC-MS/MS (at NCKU Medical College Proteomics Research Core Laboratory), and then confirmed by matching to Matrix Science database.

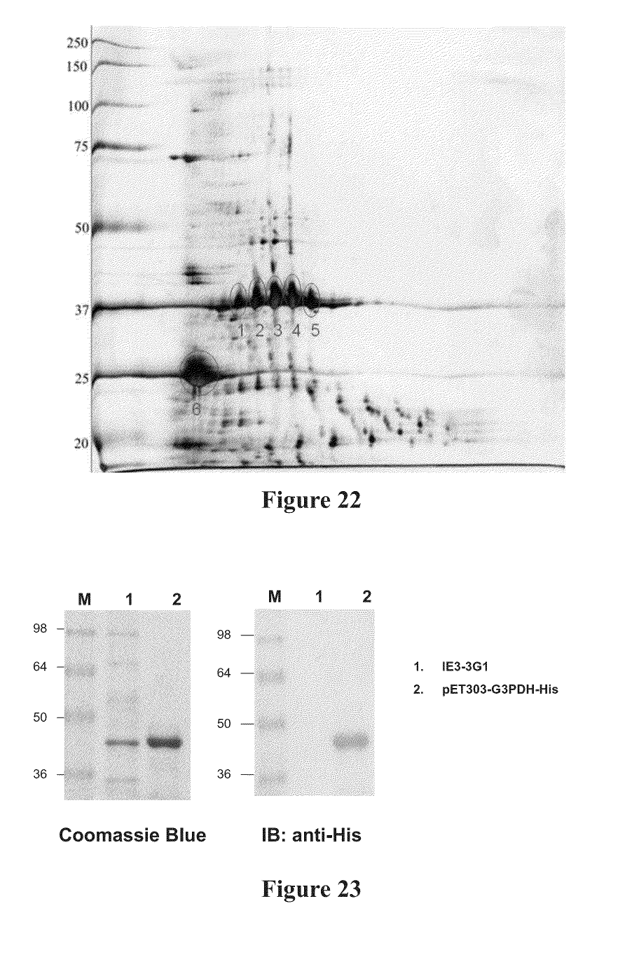

Identification of the active ingredient was done through a two-dimensional electrophoresis analysis of the sub-fraction IE3-3G1. It was found that there were six protein spots in total, including five protein spots in 40 kD and one protein spot in 25 kD. The protein was identified with LC-MS/MS and confirmed by matching them with a sequence database. Protein spots 1-5 were Glyceraldehyde 3-phosphate Dehydrogenase (G3PDH), and protein spot number 6 was Fructose-bisphosphate Aldolase (see FIG. 22). Thus, the active ingredient was identified by a two-dimensional electrophoresis analysis and LC-MS/MS. It is concluded that the active ingredient is a 40 kD protein, G3PDH.

### Example 9

### Construction of a Plasmid Comprising G3PDH Gene and Expression of G3PDH

To further confirm that G3PDH is an active ingredient in the PM-A0005 strain, the recombinant protein was expressed. First, an oligo-primer comprising restriction enzyme cut point was designed according to the DNA sequence in genome data base, amplified with PCR, and the DNA was recombined with pET303/CT-His (Invitrogen, USA) expression vector to obtain a pET303-G3PDH-His construct. The pET303-G3PDH-His construct was transformed in E. coli DH5α competent cells to replicate the construct thereof. The construct was then purified with a mini plasmid extraction kit (Viogen, USA) and confirmed by DNA sequencing.

The gene sequence of G3PDH isolated from Lactobacillus gasseri was found in the NCBI genome database (SEQ ID No: 2). The PM-A0005 strain, with a specific primer as shown in Table 5, was amplified by PCR. The amplified DNA was cut by the restriction enzymes, XbaI and XhoI. The pET303/CT-His plasmid was purified and then was cut again by the restriction enzymes, XbaI and XhoI. The G3PDH gene and the plasmid were mixed together and ligated using DNA ligase, and cloned. E. coli was proliferated to obtain pET303-G3PDH-His. Finally, G3PDH was confirmed with DNA sequencing.

TABLE 5

| | |
|---|---|
| G3PDH | 5′-GCTCT AGAAT GACAG TTARA ATTGG |
| forward | TATTA A-3′ |
| primer | (SEQ ID No: 3) |
| G3PDH | 3′-AAATA ACTTT AAACG ATRAG AAGAG |
| reverse | CTCGC C-5′ |
| primer | (SEQ ID No: 4) |

The G3PDH DNA sequence was transcribed to the amino acid sequence of protein, set forth in SEQ ID No: 1.

Next, the recombinant plasmid pET303-G3PDH-His was transformed into BL21-DE5, and the transformation was confirmed with colony PCR. The BL21-DE5 strain comprising recombinant plasmid pET303-G3PDH-His was incubated in LB culture medium at 37° C. until the OD reached 0.4, where the broth was moved to 26° C. incubation and IPTG was added to induce expression of recombinant protein. Then, the strain was centrifuged for 20 minutes at 4,000 rpm and 4° C. Lysosome (1 mg/ml) was used to dissolve the strain, and the precipitation was removed by centrifuging at 22,500 g and 4° C. for 30 minutes. An affinity column, with nickel ions that can bind specifically to the His tag, was used to purify the recombinant protein in the supernatant. The protein concentration was then measured with a spectrophotometer at 280 nm, and stained with Coomassie Blue. The purity of the protein was determined by a western blot with the histidine antibody, and it showed that there was a high expression of G3PDH recombinant protein, purer G3PDH-His recombinant protein can be obtained, and confirmed that the recombinant protein contained the His-tag (see FIG. 23).

### Example 10

### Effect of G3PDH in Stimulating Immune Cells to Secrete IL-12 and IFN-γ

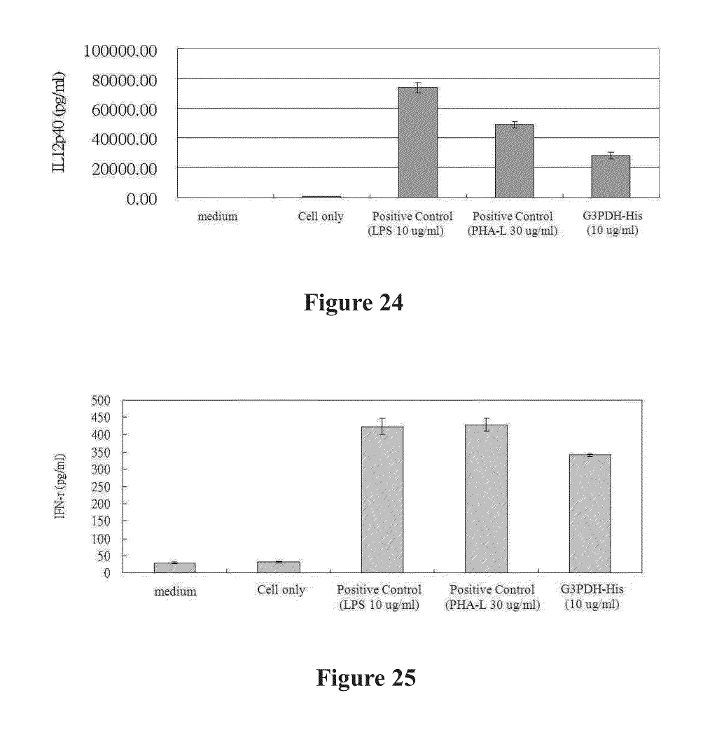

The G3PDH recombinant protein was co-cultured with mice dendritic cells to evaluate the effect in immune regulation, wherein the cells co-cultured with LPS and PHA were used as the positive controls respectively, and the cells without stimulus were used as the control group. The results are shown in Table 6. It is demonstrated that G3PDH was able to induce mice dendritic cells to generate cytokine IL-12 (28235.06 pg/ml, on average), which is higher than that of the control group (cells only) by 44 times (see FIG. 24).

TABLE 6
The data of G3PDH recombinant protein in stimulating
mice dendritic cells to generate IL12p40.

| | | cell | LPS | PHA | G3PDH-His |
|---|---|---|---|---|---|
| | medium | only | 10 ug/ml | 30 ug/ml | 10 ug/ml |
| 1st | −0.30 | 632.06 | 71382.12 | 47440.94 | 29735.06 |
| 2nd | 0.29 | 653.23 | 76382.12 | 50323.29 | 26735.06 |
| Avg. | 0.00 | 642.64 | 73882.12 | 48882.12 | 28235.06 |
| STDEV | 0.42 | 14.97 | 3535.53 | 2038.13 | 2121.32 |

The G3PDH recombinant protein was co-cultured with mice spleen cells to evaluate the effect of immune regulation, wherein the cells co-cultured with LPS and PHA were used as the positive controls respectively, and the cells without stimulus were used as the control group. The results are shown in Table 7. It is evidenced that G3PDH was able to induce mice spleen cells to generate cytokine IFN-γ (341.32 pg/ml on average), which is higher than that of the control group by 10 times (see FIG. 25).

TABLE 7
Results of G3PDH recombinant protein in stimulating
mice spleen cells to generate IFN-γ.

| | RPMI | cell | LPS | PHA | G3PDH |
|---|---|---|---|---|---|
| | 1640 | only | 10 ug/ml | 30 ug/ml | 10 ug/ml |
| 1st | 28.42 | 33.68 | 407.37 | 442.63 | 343.68 |
| 2nd | 31.05 | 30.00 | 441.58 | 414.74 | 338.95 |
| Avg. | 29.74 | 31.84 | 424.47 | 428.68 | 341.32 |
| STDEV | 1.86 | 2.61 | 24.19 | 19.72 | 3.35 |

In summary, it is evidenced by the results of the in vitro and in vivo experiments that either G3PDH, or the PM-A0005 strain, the extract, fraction, or sub-fraction of the PM-A0005 strain is effective in stimulating immune cells to secrete IL-10, IL-12, and IFN-γ. Then, the Th1 immune response can be regulated and the excessive Th2 immune response due to allergies can be improved, and the proliferation of Th17 cell can be affected; thus, G3PDH, or the PM-A0005 strain, the extract, fraction, sub-fraction thereof is able to regulate immune response. In addition, the active ingredient, G3PDH, or the composition comprising G3PDH, has an effect in decreasing airway resistance in Der p allergen-sensitized mice and inhibiting cell infiltration and inflammation in the lungs, ameliorating the thickening of the epidermal layer and dermis layer, and immune cell infiltration. Given the above, they are potent to be a medicament for treating or preventing allergic diseases including but not limited to asthma and atopic dermatitis.

It is believed that a person of ordinary skill and knowledge in the art to which the present invention belongs can utilize the present invention to its broadest scope based on the descriptions herein with no need of further illustration. Therefore, the descriptions and claims as provided should be understood as of demonstrative purpose, instead of limitative in any way, to the scope of the present invention.

I/We claim:

1. A method for preventing or treating an allergic disease in a subject comprising administering the subject with a composition comprising a therapeutically effective amount of glyceraldehyde-3-phosphate Dehydrogenase (G3PDH) or the functional variant or fragment thereof as an active ingredient to reduce allergic response.

2. The method according to claim 1, wherein the allergic disease is asthma.

3. The method according to claim 1, wherein the allergic disease is atopic dermatitis.

4. The method according to claim 1, wherein the G3PDH has the amino acid sequence set forth in SEQ ID No: 1.

5. The method according to claim 1, wherein the G3PDH is obtained by isolating from Lactobacillus gasseri PM-A0005 strain (PM-A0005 strain), the extract, fraction, or sub-fraction thereof; wherein the PM-A0005 strain was deposited in the China Center for Type Culture Collection (CCTCC) with Deposit No: M 207039.

6. The method according to claim 5, wherein the extract is obtained by decomposing the PM-A0005 strain by lysozyme and precipitating cytoplasmic protein by ammonium sulfate.

7. The method according to claim 6, wherein the extract is obtained by precipitating cytoplasmic protein by the ammonium sulfate in a concentration more than 0% but less than 25%, or a concentration of 50-75%.

8. The method according to claim 5, wherein the fraction is obtained by further purification of the extract through ion-exchange chromatography.

9. The method according to claim 8, wherein the fraction is selected from the group consisting of IE1-2, IE1-3, IE3-2, and IE3-3.

10. The method according to claim 5, wherein the sub-fraction is obtained by further purification of the fraction through gel filtration chromatography.

11. The method according to claim 10, wherein the sub-fraction is IE3-3G1.

12. The method according to claim 5, wherein the composition is a pharmaceutical composition or a food composition further comprising a physiological acceptable excipient or diluent.

13. The method according to claim 1, wherein the composition is manufactured from the PM-A0005 strain), the extract, fraction, or sub-fraction thereof, together with a physiological acceptable excipient or diluent in a form of a pharmaceutical composition or a food composition.

14. A method for treating or preventing atopic dermatitis in a subject comprising administering the subject with a composition comprising a therapeutically effective amount of G3PDH or the functional variant or fragment thereof as an active ingredient.

15. The method according to claim 14, wherein the G3PDH has the amino acid sequence set forth in SEQ ID No: 1.

16. The method according to claim 14, wherein the G3PDH is obtained by isolating from Lactobacillus gasseri PM-A0005 strain (PM-A0005 strain), the extract, fraction, or sub-fraction thereof, wherein the PM-A0005 strain was deposited in the China Center for Type Culture Collection (CCTCC) with Deposit No: M 207039.

17. The method according to claim 14, wherein the composition is a pharmaceutical composition or a food composition further comprising a physiological acceptable excipient or diluent.

18. A method for treating or preventing atopic dermatitis in a subject comprising administering the subject with a composition comprising a therapeutically effective amount of the PM-A0005 strain, the extract, fraction, or sub-fraction thereof; wherein the PM-A0005 strain was deposited in the China Center for Type Culture Collection (CCTCC) with Deposit No: M 207039.

19. The method of claim 18, wherein the composition comprises the PM-A0005 strain.

20. The method of claim 18, wherein the composition comprises the extract of the PM-A0005 strain.


SEQUENCE LISTING (4 sequences)

SEQ ID NO: 1 - protein, 346 aa, Lactobacillus gasseri
MTVRIGINGFGRIGRLAFRRIMDLGEKSSDIEVVAINDLTTPALLAHLLKYDSTHGTFNHEVSATDDSIVVDGKKYRVYAEPQAQNIPWVKNDGVDFVLECTGFYTSKAKSEAHLKAGAKRVLISAPAGSDLKTIVYGVNDDTLTADDKIVSAGSCTTNSLAPMVNALQKEFGIEVGTMTTIHAYTSTQMLLDGPVRGGNLRSARAAAINIIPHSTGAAKAIGLVVPELNGKLNGHAQRVPVPDGSVTDLVSILSKDVTADEVNEAVKKYESPSFAYNDHNIVSSDVLGMTAGSIFDPTQTMVTTAGDKQLVKTVAWYDNEYSFTCQMVRTLLKFATLLEHHHHHH

SEQ ID NO: 2 - DNA, 1017 bp, lactobacillus gasseri
ttaaagagtagcaaatttcaataaagtacgaaccatttggcaagtgaatgagtattcgttgtcgtaccaagcaacagtcttaactaattgcttgtcacctgcagtagttaccatggtttgagttgggtcaaagattgaaccagcagtcatacctaaaacgtcgctagaaacgatgttgtggtcgttgtatgcaaatgaaggactttcgtacttcttaactgcttcgttaacttcgtcagcagttacatccttgcttaagattgaaaccaattcagttacagaaccatctggaactggaacacgttgtgcgtgaccattcaacttaccgttcaattctggaacaacaagaccaatagccttagcagcaccagttgagtgaggaataatgttgatagctgcagcacgagcagaacgtaagttaccaccacgtacaggaccatctaaaagcatttgagttgaagtgtaagcgtggatagtagtcatagtaccaacttcaatgccgaattccttttgtaaagcattaaccattggtgctaatgagttagtagtacatgaaccagctgaaacaatcttgtcgtcagcagtcaaagtatcatcgtttacgccgtaaacaatggtcttcaagtctgaaccagcaggtgcagaaattaatacacgctttgcaccagccttaaggtgagcttctgacttagctttgctagtgtagaaaccagtacattcaagaacgaagtcaacaccatcgttcttaacccaaggaatgttttgtgcttgtggttcagcgtaaacacggtacttcttaccgtcaactacgattgagtcatcagttgctgaaacttcgtggttgaaagtaccatgagttgagtcgtacttaagtaagtgagctaaaagtgctggagtagtcaagtcgttgattgcaacaacttcaatatctgaagacttttcgcctaaatccataatacgacggaatgctaaacgaccaatacggccgaaaccgttaataccaattttaactgtcat

SEQ ID NO: 3 - DNA, 31 bp, Artificial Sequence
  Primer
gctctagaatgacagttaraattggtattaa

SEQ ID NO: 4 - DNA, 31 bp, Artificial Sequence
  Primer
aaataactttaaacgatragaagagctcgcc